SEQUENCE LISTING (20 sequences)

SEQ ID NO: 1 - DNA, 20 bp, Artificial Sequence
  synthetic construct
aactttggcattgtggaagg

SEQ ID NO: 2 - DNA, 20 bp, Artificial Sequence
  Synthetic construct
acacattgggggtaggaaca

SEQ ID NO: 3 - DNA, 22 bp, Artificial Sequence
  Synthetic construct
cagagaggccaacctacttttc

SEQ ID NO: 4 - DNA, 21 bp, Artificial Sequence
  Synthetic construct
atgcgccccaaggtcatattt

SEQ ID NO: 5 - DNA, 20 bp, Artificial Sequence
  Synthetic construct
ggaagtgaacaaagggacca

SEQ ID NO: 6 - DNA, 20 bp, Artificial Sequence
  Synthetic construct
tccctgcagcttcagtacct

SEQ ID NO: 7 - DNA, 22 bp, Artificial Sequence
  Synthetic construct
ccttgatcccatccaccccgga

SEQ ID NO: 8 - DNA, 22 bp, Artificial Sequence
  Synthetic construct
cccggacacatcaagcacctgg

SEQ ID NO: 9 - DNA, 19 bp, Artificial Sequence
  Synthetic construct
caggcggtgcctatgtctc

SEQ ID NO: 10 - DNA, 22 bp, Artificial Sequence
  Synthetic construct
cgatcaccccgaagttcagtag

SEQ ID NO: 11 - DNA, 23 bp, Artificial Sequence
  Synthetic construct
tagtccttcctaccccaatttcc

SEQ ID NO: 12 - DNA, 21 bp, Artificial Sequence
  Synthetic construct
ttggtccttagccactccttc

SEQ ID NO: 13 - DNA, 20 bp, Artificial Sequence
  Synthetic construct
tcttagacggccttgcagat

SEQ ID NO: 14 - DNA, 20 bp, Artificial Sequence
  Synthetic construct
agggactacccctgaaggaa

SEQ ID NO: 15 - DNA, 20 bp, Artificial Sequence
  Synthetic construct
gaaacagcccgagtcagaag

SEQ ID NO: 16 - DNA, 20 bp, Artificial Sequence
  Synthetic construct
gagcctgccattcttacagg

SEQ ID NO: 17 - DNA, 20 bp, Artificial Sequence
  Synthetic construct
gcgccaagctatcagtatcc

SEQ ID NO: 18 - DNA, 20 bp, Artificial Sequence
  Synthetic construct
tcagccatctgagcacattc

SEQ ID NO: 19 - DNA, 23 bp, Artificial Sequence
  Synthetic construct
ctcaggctgtatgtcagatgttc

SEQ ID NO: 20 - DNA, 22 bp, Artificial Sequence
  Synthetic construct
aagcgagtcacagagattggtc

(19) United States
(12) Patent Application Publication
(10) Pub. No.: US 2019/0241962 A1
(43) Pub. Date: Aug. 8, 2019

(54) GENETIC SUSCEPTIBILITY DIAGNOSIS AND TREATMENT OF MENTAL DISORDERS

(71) Applicant: Sharon Anavi-Goffer, Oranit (IL)

(72) Inventor: Sharon Anavi-Goffer, Oranit (IL)

(21) Appl. No.: 16/338,654

(22) PCT Filed: Oct. 2, 2017
(86) PCT No.: PCT/IB2017/001348

§ 371 Date: Apr. 1, 2019

Related U.S. Application Data
(60) Provisional application No. 62/403,147, filed on Oct. 2, 2016.

(51) Int. Cl. C12Q 1/6883; C12Q 1/6897

(52) U.S. Cl. CPC C12Q 1/6883; C12Q 1/6897; C12Q 2600/106; C12Q 2600/156; C12Q 2600/118

(57) ABSTRACT

Aspects of the invention provide methods of screening for a mental disease selected from schizophrenia, psychosis and phencyclidine abuse and addiction in a subject or a subject population, diagnosing schizophrenia, psychosis and phencyclidine abuse and addiction in a subject by determining the magnitude of expression of at least one gene and providing tools for selection of a treatment and a list of therapeutic agents for the treatment of schizophrenia, psychosis and phencyclidine abuse or addiction based on said screening and diagnosis of a human or a nonhuman animal.

### CROSS REFERENCE TO RELATED APPLICATIONS

This application claims priority to U.S. Provisional Patent Application Ser. No. 62/403,147, filed on Oct. 2, 2016, the entire content of which is hereby incorporated by reference in its entirety.

### FIELD OF THE INVENTION

Aspects of the invention provide methods of screening and treating a mental disease such as schizophrenia, psychosis and phencyclidine abuse and addiction in a subject or a subject population.

### BACKGROUND OF THE INVENTION

Schizophrenia is a chronic psychotic disease that affects 1% of the world population. The causes of schizophrenia are partly a result of genetic changes but also of environmental factors.

Clinical studies indicate that inflammatory processes are involved in the pathophysiology of schizophrenia. Specifically, abnormal levels of pro-inflammatory cytokines TNF-α and IL-6 were found in patients with schizophrenia. In recent years, it became apparent that increased use of cannabis is a risk factor associated with the onset of psychosis especially in people with a genetic predisposition to schizophrenia. Accordingly, a number of studies have found significant changes in the endocannabinoid system in patients with schizophrenia and in models of schizophrenia in laboratory animals. Changes in the endocannabinoid system include genetic polymorphisms, changes in levels of cannabinoid receptors, changes in levels of endocannabinoids and changes in levels of enzymes that regulate the levels of endocannabinoids. Some of these changes were found in the DNA, central nervous system and some in the blood. Since these changes were found in various studies it remains unclear whether these changes occur in the same area of the brain and whether they occur all at once.

Schizophrenia is often chronic, characterized by deterioration of social contact, cognitive deficits, anxiety and depression, resulting in suicide in about 10% of the schizophrenic population (Ross et al., 2006). Reduced glutamate concentrations and glutamate receptor densities have been found in the brains of schizophrenics post-mortem giving rise to the ‘glutamate theory’ in the pathophysiology of schizophrenia (Ross et al., 2006). Indeed, NMDA receptor antagonists such as phencyclidine, ketamine and dizocilpine were found to induce psychotic alterations in healthy humans which resemble those of schizophrenia (Ross et al., 2006). Based on these findings animal models of schizophrenia were established using NMDA antagonists (Mouri et al., 2012).

The endocannabinoid ligands, their transporter, receptors (cannabinoid type one CB1 receptor and cannabinoid type two CB2 receptor), anabolic enzymes and degradative enzymes constitute the major parts of the endocannabinoid system. CB1 receptors are present at high concentrations in many major brain structures including the cortex, hippocampus, basal nuclei and amygdala as well as in organ systems including the immune, reproductive systems and gastrointestinal tract (review by Pertwee et al., 2010). Under normal physiological conditions, the cannabinoid CB2 receptor is most abundant in the immune system and bones (reviewed by Atwood et al., 2010). However, CB2 receptor immunoreactivity was also visualized in major parts of the rat brain including the cerebral cortex, hippocampus and cerebellum, brainstem cells of mice, rat and ferret and human perivascular microglia cells (Atwood et al., 2010).

### SUMMARY OF THE INVENTION

Aspects of the invention provide methods of screening for a mental disease selected from schizophrenia, psychosis and phencyclidine abuse or addiction in a subject or a population, diagnosing schizophrenia, psychosis and phencyclidine abuse or addiction in a subject or a population by determining the magnitude of expression of at least one gene causing the mental disease and providing tools for selection of active agents and a list of suitable active agents for the treatment of schizophrenia, psychosis and phencyclidine abuse or addiction based on said screening and diagnosis of a human or a nonhuman subject.

The present disclosure discovered differences between the expression levels of these genes in the left vs. right hemispheres (lateralization).

Based on the lateralization discovery of this invention, women and men will receive different combinations of therapeutic agents. As such, drug treatment will be useful targeting specific brain region, a specific hemisphere and tailored to gender and age.

According to aspects of the invention, patients that have mutations which results in absolute inactive protein of one the relevant genes shall not receive specific treatments, i.e. beta-caryophyllene (BCP) will not be recommended for a subject with a mutation that leads to inactive CB1 receptor and CB2 receptor proteins. There is provided a therapeutic composition comprising BCP and/or HU-308 and a pharmaceutically effective carrier for use in treating schizophrenia, psychosis and PCP abuse or addiction in patients diagnosed according to aspects of the invention.

Other drugs used according to aspects of the invention include a gene inhibitor selected from an antisense oligonucleotide, a nucleic acid molecule, an interfering RNAs (RNAi) selected from a small interfering RNA (siRNA), a micro interfering RNA (miRNA), an RNA-induced transcriptional silencing (RITS), a ribozyme and combinations thereof, a gene enhancer selected from, a short DNA enhancer, an eRNA enhancer molecule and combinations thereof, a gene modulator selected from a non-coding RNA transcripts, a small molecule promoter modulators, a CB2 selective agonist selected from BCP and HU-308, a FAAH enhancer, a MGL enhancer, rosmarinic acid and combinations thereof, an antibody selected from whole antibody, humanized antibody, chimeric antibody, Fab fragment, Fab′ fragment, F(ab′)2 fragment, single chain Fv fragment, diabody and combinations thereof.

A transgenic, knockout or conditional nonhuman animal comprising stably integrated in its genome or a conditional/site-directed mutation/tissue-specific mutation, selected from the group a gene encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors are also provided. Transgenic, knockout or conditional nonhuman animals can be used in methods of screening, selecting, and suitability determination of candidate therapeutic active agents.

Kits and instructions according to aspects of the invention are provided, comprising a custom array selected from a gene array, a probe array, a protein array, an array comprising a therapeutic agent, a nucleic acid molecule, a cell or a kit component which expresses a patient's mutation, to at least one of the genes selected above and their combination thereof.

### BRIEF DESCRIPTION OF THE FIGURES

The presently disclosed embodiments will be further explained with reference to the drawings. The drawings are not necessarily in scale, with emphasis instead generally placed upon illustrating the principles of the presently disclosed embodiments.

FIGS. 1A-1C show ambulation behavior in mice. In Sabra female mice, compared with the control group, ambulation behavior was reduced in the PCP-treated group (A). In Sabra male mice, ambulation behavior was elevated in the PCP-treated group (B).Treatment with BCP reversed altered behavior (A-females, B-males). In CB1 receptor knockout mice, compared with the control group, ambulation behavior was significantly elevated in the PCP-treated group. Treatment with BCP did NOT reverse this behavior (C-females).

FIGS. 2A-D show IL-6 mRNA level in the cortex. Compared with the control group (A and C), the mRNA level of IL-6 in female mice was reduced (B) and in the male mice was elevated (C) in the PCP-treated group in the right cortex but not in the left cortex. BCP did not alter gene expression of IL-6 in female but reversed the effect of PCP in male mice (B and D, respectively).

FIGS. 3A-D show CB1 receptor mRNA level in the cortex. Compared with the control group (A and C), the mRNA level of CB1 receptor in females (B) and males (D) was elevated in the PCP-treated group in the RIGHT cortex but not in the left cortex. BCP did not alter gene expression of CB1 receptor in the cortex of female or male mice (B and D, respectively).

FIGS. 4A-D show CB2 receptor mRNA level in the cortex. Compared with the control group (A and C), the mRNA level of CB2 receptor in females was reduced (B) in the PCP-treated group in the RIGHT cortex. There was no change in the left cortex of PCP-treated group vs. the control group. BCP reversed the effect of PCP on CB2 receptor on gene expression in the right cortex of female or male mice (B and D, respectively).

FIGS. 5A-D show ABHD6 mRNA level in the cortex. Compared with the control group (A and C), the mRNA level of ABHD6 in females (B) but not in males (C) was reduced in the PCP-treated group in the LEFT cortex but not in the right cortex. BCP did not alter gene expression of ABHD6 in female or male mice (B and D, respectively).

FIGS. 6A-D show MGL mRNA level in the cortex. Compared with the control group (A and C), the mRNA level of MGL in females (B) but not in males (C) was reduced in the PCP-treated group in the LEFT cortex but not in the right cortex. The reduction in MGL mRNA level was unexpected. BCP did not alter gene expression of MGL in female mice (B and D, respectively).

FIGS. 7A-D show FAAH mRNA level in the cortex. Compared with the control group (A and C), the mRNA level of FAAH in females (B) but not in males (C) was reduced in the PCP-treated group in the LEFT cortex but not in the right cortex. The reduction in FAAH mRNA level was unexpected. BCP did not alter gene expression of FAAH in female mice (B and D, respectively).

FIGS. 8A-I show comparison between females and males of mRNA level of GAPDH, TNF-alpha and IL-6 in the cortex and brain stem. IL-6 delta Ct level of mRNA is lower in males compared with females in the right and left cortex and in the brain stem.

FIGS. 9A-F show comparison between females and males of mRNA level of CB1 receptor and CB2 receptor in the cortex and brain stem. Comparison of between females and males revealed that delta Ct level of mRNA level of and CB1 receptor is significantly lower in males compared with female mice in the RIGHT cortex. While CB2 receptor delta Ct level of mRNA in males is higher than this of females in the LEFT cortex.

FIGS. 10A-I show comparison between females and males of mRNA level of ABHD6, MGL and FAAH in the cortex and brain stem. Comparison of mRNA level between females and males revealed that for genes ABHD6, MGL and FAAH the delta Ct level of mRNA in males is significantly higher than this of females in the left cortex.

FIGS. 11A-G show comparison of mRNA level between the left and right cortices of females. In females, comparison of mRNA level between left vs. right cortex revealed that the delta Ct level of mRNA for genes FAAH and ABHD6 is higher the right cortex than in the left cortex.

FIGS. 12A-G show comparison of mRNA level between the left and right cortices of males. In males, comparison of mRNA level between left vs. right cortex revealed that there are no differences in the expression level of the selected genes.

FIGS. 13A-D show NAPE-PLD mRNA level in the cortex. Compared with the control group (A and B), the mRNA level of the enzyme NAPE-PLD in female mice was reduced in the PCP-treated group in the right and left cortices. BCP did not reverse this effect. Compared with the control group (C and D), the mRNA level of the enzyme NAPE-PLD in male mice was not altered in the PCP-treated group. BCP did not affect the expression level of NAPE-PLD mRNA in male mice cortex (B and D, respectively).

FIGS. 14A-D GAD67 mRNA level in the cortex. Compared with the control group (A and B), the mRNA level of the enzyme GAD67 in female mice was reduced in the PCP-treated group in the right and left cortices. BCP did not reverse this effect. Compared with the control group (C and D), the mRNA level of the enzyme GAD67 in male mice was not altered in the PCP-treated group. BCP did not affect the expression level of GAD67 mRNA in male mice cortex (B and D, respectively).

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

Definitions

The term “targeted genes” means genes that were found to be affected in models or patients.

The term “lateralization” refers to the way some areas in brain and spinal cord function, neural functions and cognitive processes are significantly stronger or tend to be more dominant in one hemisphere than the other, i.e. right hemisphere vs. left hemisphere.

The term ‘ribozymes’ refers to RNAs capable of catalyzing RNA cleavage reactions, and some can be designed to specifically cleave a particular target mRNA. Ribozyme methods include exposing a cell to, inducing expression in a cell, etc. of such RNA ribozyme molecules.

The term “activity of a target RNA” (preferably mRNA) species, specifically its rate of translation, can be inhibited by the application of antisense nucleic acids.

The term “nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” refers to nucleic acids which are complementary to a specific DNA or RNA sequence, capable of hybridizing to a sequence specific portion of the target RNA, e.g., its translation initiation region by virtue of some sequence that is complementary to a coding and/or non-coding region. The antisense nucleic acid can be oligonucleotides that are double-stranded or single-stranded, RNA or DNA or a modification or derivative thereof, which can be produced intracellularly by transcription of exogenous, introduced sequences in controllable quantities sufficient to perturb translation of the target RNA.

The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation.

The designation “negative” is sometimes used in reference to the antisense strand, and “positive” is sometimes used in reference to the sense strand. As contemplated herein, antisense oligonucleotides, triple helix DNA, RNA aptamers, RNAi, ribozymes and double or single stranded RNA are directed to a nucleic acid sequence of a gene disclosed in Table 2 such that the nucleotide sequence of the gene chosen will produce gene-specific inhibition of gene expression. For example, knowledge of the target gene nucleotide sequence may be used to design an antisense molecule which gives strongest hybridization to the mRNA. Similarly, ribozymes can be synthesized to recognize specific nucleotide sequences and cleave them (Cech. J. Amer. Med Assn. 260:3030 (1988)). Techniques for the design of such molecules for use in targeted inhibition of gene expression are well known to one of skill in the art.

The above described techniques are emerging as an effective means for reducing the expression of specific gene products and may therefore prove to be uniquely useful in a number of therapeutic, diagnostic and research applications for the modulation of genes that are dysregulated or upregulated in schizophrenic patients or nonhumans.

A “therapeutically effective amount” is the amount of any type of therapeutic agent that is sufficient to treat and/or ameliorate the schizophrenia or schizophrenia-like effects, including but not limited to hallucinations, dilutions, emotional effects, cognitive effects, attention effects, social effects.

The term “therapeutic agent” as used herein describes any molecule, e.g. protein, enzyme, carbohydrate, metal or organic compound, with the capability of affecting the molecular and clinical phenomena associated with schizophrenia. Generally, a plurality of assay combinations may be run in parallel with different agent concentrations to obtain a differential response to the various concentrations. Typically, one of these concentrations serves as a negative control, i.e. at zero concentration or below the level of detection.

The terms “active agent”, “agent”, “therapeutic agent” and “API” are used interchangeably.

The term “beta-caryophyllene (BCP)” refers to E-BCP (CAS 87-44-5) and/or Z-BCP (CA 118-65-0) isomers of beta-caryophyllene in the presence or absence of derivatives such as BCP oxide (CAS 1139-30-6) and minor sesquiterpenes such as alpha-humulene (CAS 6753-98-6), copaene (CAS 3856-25-5) and eugenol (CAS 97-53-0).

The term E-BCP refers to substantially pure E-BCP, comprising 95%, 96%, 97%, 98% or more of E-BCP.

The term “antibody” refers to intact molecules as well as fragments thereof, such as Fa, F(ab′)2j and Fv, which are capable of binding the epitopic determinant. Antibodies that bind polypeptides of interest can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat, a rabbit or a pig).

The term “humanized antibody” as used herein, refers to antibody molecules in which amino acids have been replaced in the non-antigen binding regions in order to more closely resemble a human antibody, while still retaining the original binding ability.

The term “subject” refers to any human or nonhuman organism.

The terms “altering the magnitude of expression of a gene” or “alteration of the magnitude of expression of a gene' or “departure from the baseline magnitude of expression of a gene”, are used interchangeably.

The term “protein' in the context of this disclosure refers to the translated protein of the transcription of a selected gene.

Aspects of the present invention disclose methods for detecting alterations in mRNA expression with a view to screen for genes dysregulated in various mental diseases and disorders. In organisms for which the complete genome is known, it is possible to analyze the transcripts of all genes within a cell, tissue, organ or whole body. With some organisms, such as the human organism, for which there is an increasing knowledge of the genome, it is possible to simultaneously monitor large numbers of genes within a cell. DNA, RNA or protein microarray analysis is a technique that permits the quantitative measurement of the transcriptional expression of several thousand genes or proteins simultaneously. This technique permits one to generate profiles of gene or protein expression pattern in both patients suffering from schizophrenia and/or psychosis and control individuals.

Using these techniques, the determination of abnormal levels of gene or protein expression according to this invention provides a diagnostic indication that therapeutic treatment is needed.

Effective techniques for reducing the expression of specific gene products are disclosed. Diagnostic and/or modulation of the level of genes that are related to the endocannabinoid system which are found dysregulated in mouse model of schizophrenia are novel approaches to diagnose and/or treat mental diseases selected from schizophrenia psychosis and phencyclidine (PCP) abuse or addiction, as well as other NMDA antagonists-induced abuse or addiction such as ketamine and dizocilpine.

In some embodiments, there is provided a method for screening for subjects susceptible to a mental disease selected from schizophrenia, psychosis and PCP addiction or abuse in a population which includes determining, in members of the population, the magnitude of expression in a subject sample of a gene selected from the group consisting of the gene encoding the IL-6, gene encoding the TNF-alpha (TNF-α), the gene encoding the glutamate decarboxylase/glutamic acid decarboxylase (GAD) genes particularly the gene encoding GAD67 enzyme, the gene encoding the CB1 receptor, the gene encoding the CB2 receptor, the gene encoding for the GPR55 receptor, the gene encoding fatty acid amide hydrolase (FAAH) enzyme, the gene encoding monoacylglycerol lipase (MGL) enzyme, the gene encoding the α/β-hydrolase domain containing 6 (ABHD6 or ABDH6) enzyme, the gene encoding the α/β-hydrolase domain containing a12 enzyme (ABHD12 or ABDH12) enzyme, the gene encoding the α/β-hydrolase domain containing 4 (ABHD4 or ABDH4) enzyme, the gene encoding the sn-1-diacylglycerol lipase alpha (DAGL-alpha) enzyme, the gene encoding the sn-1-diacylglycerol lipase beta (DAGL-beta) enzyme, the gene encoding N-acyl phosphatidylethanolamine phospholipase D (NAPE-PLD) enzyme, the gene encoding the phosphodiesterase 1 (GDE1) enzyme, the gene encoding for phospholipase C (PLC) enzyme, the gene encoding for phospholipase D (PLD) enzyme, the genes encoding 5-hydroxytryptamine (5-HT) receptors (HTR) and comparing the magnitude of expression to a baseline magnitude of expression of the gene, wherein increased or decreased expression according to a selected gene and/or combination of genes indicates the presence of schizophrenia, psychosis or PCP abuse or addiction. The sample (one cell or more or a piece of tissue) may be obtained from a body fluid selected from cerebrospinal fluid (CSF), blood, saliva, lymphatic fluid, urine or feces, or from a body organ selected from epithelial cells, spleen, skin or hair according to Example 3.

In some embodiments, the sample is taken from a specific left or right side of the brain or spinal cord.

In some embodiments, the sample is taken from the prefrontal cortex.

In some embodiments, the sample is taken from the brain stem.

In some embodiments, the sample is taken from the hippocampus.

In some embodiments, the sample is taken from the spinal cord.

According to some embodiments, the population is human.

In some embodiments, the population is nonhuman.

According to some embodiments, the subject is human.

In some embodiments, the subject is nonhuman.

In another aspect, there is provided a method for diagnosing schizophrenia, psychosis or PCP abuse or addiction in a subject which includes determining the magnitude of expression of a gene selected from the group consisting of the gene encoding the GAD67, the gene encoding the IL-6, gene encoding the TNF-alpha, gene encoding the CB1 receptor, the gene encoding the CB2 receptor, the gene encoding the GPR55 receptor the gene encoding FAAH enzyme, the gene encoding MGL enzyme, the gene encoding the ABHD6, the gene encoding the ABHD12, the gene encoding the ABHD4, the gene encoding the DAGL-alpha, the gene encoding the DAGL-beta, the gene encoding NAPE-PLD, the gene encoding the GDE1, the gene encoding for PLC, the gene encoding for PLD, the genes encoding HTR (the genes encoding 5-HT receptors) and comparing the magnitude of expression to a baseline magnitude of expression of the gene or its protein, wherein decreased gene expression of selected genes indicates the presence of schizophrenia. In some embodiments, the subject is human. In other embodiments, the subject is nonhuman. In some embodiments, the population is human. In another preferred embodiment, the population is nonhuman.

In some embodiments, there is provided a method for treating schizophrenia of all symptoms, onset at any age.

The present invention uncovered that some genes are upregulated in schizophrenic-like mice while others were found to be downregulated compared to baseline or normal levels. The level of some other genes was not changed. The method of treatment of this invention takes this finding into account.

In some embodiments, the subject is human. In other embodiments, the subject is nonhuman.

In another aspect, there is provided a method for treating schizophrenia in a subject in need thereof which includes decreasing the expression of a gene selected from the group consisting of the gene encoding the GAD67 (GAD1), the gene encoding the IL-6, gene encoding the TNF-alpha, gene encoding the CB1 receptor, the gene encoding the CB2 receptor, the gene encoding the GPR55 receptor, the gene encoding FAAH enzyme, the gene encoding MGL enzyme, the gene encoding the ABHD6, the gene encoding the ABHD12, the gene encoding the ABHD4, the gene encoding the DAGL-alpha, the gene encoding the DAGL-beta, the gene encoding NAPE-PLD, the gene encoding the GDE1, the gene encoding for PLC or the gene encoding for PLD, the genes encoding HTR by administering to the subject an expression-lowering amount of a ribozyme which cleaves the RNA associated with expression of the gene.

In some embodiments, the subject in the population is human. In some embodiments, the subject in the population is nonhuman.

In some embodiments, there is provided a method for treating schizophrenia which includes decreasing the amount of the genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors in a subject in need thereof, by administering to the subject a decreasing effective amount of antibody against each one of the targets or a multi-target antibody to target one or more of the selected proteins.

In some embodiments, there is provided a method of screening for drugs or therapeutic active agents which are suitable for the treatment of a mental disease selected from schizophrenia, psychosis and PCP abuse or addiction, which includes operatively linking a reporter gene which expresses a detectable protein to a regulatory sequence for a gene selected from the group consisting of the gene encoding the GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors to produce a reporter construct, transfecting a cell with the reporter construct, exposing the transfected cell to a test drug, and comparing the level of expression of the reporter gene after exposure to the test drug to the level of expression before exposure to the test drug, wherein an altered level of expression after exposure which is similar to the level of a healthy subject is indicative of a compound useful for the treatment of schizophrenia.

In some embodiments, there is provided a transgenic nonhuman animal which stably includes in its genome an increased/decreased copy number of a gene selected from the group consisting of the gene encoding the GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors, wherein the gene is expressed at abnormal or changed levels (higher or lower) than baseline levels and the animal exhibits abnormal behavior.

In another embodiment, there is provided a transgenic animal which includes in its genome a gene selected from the group consisting of the gene encoding the GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors, wherein the expression of the gene is increased by at least one alteration in regulatory sequences of the gene such that the gene is expressed at higher than baseline levels and the animal exhibits abnormal behavior. In a preferred embodiment, the one or more alterations comprises substitution of a promoter having a higher rate of expression than the native promoter of the gene. In some embodiments, the promoter is an inducible promoter.

In another embodiment, a transgenic nonhuman knockout animal is provided whose genome includes a homozygous disruption in one or more genes selected from the group consisting of the gene encoding the GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors, wherein said homozygous disruption prevents the expression of the gene, and wherein said homozygous disruption results in the transgenic knockout animal exhibiting decreased expression levels of the one or more genes as compared to a wild-type animal.

In another embodiment, a transgenic conditional mutant nonhuman animal is provided whose genome includes a homozygous disruption at a specific brain region, in one or more brain regions, in one or more genes selected from the group consisting of the gene encoding the GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors, wherein said homozygous disruption prevents or enhances the expression of the gene, and wherein said homozygous disruption results in the transgenic conditional mutant animal exhibiting abnormal or changed (decreased or increased) expression levels of the one or more genes as compared to a wild-type animal.

In another embodiment, a nonhuman transgenic/knockout/conditional animal is provided with at least one gene mutation of a selected patient/subject, stably integrated in its genome or a conditional/site-directed mutation/tissue-specific mutation, selected from the group consisting of genes encoding GAD67, IL-6, TNF-alpha, CB1 receptor CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof, wherein said expression of the combination of the genes is decreased/enhanced by one or more alterations in regulatory sequences/conditional controlling mechanisms, or a regulatory sequences of the gene, wherein said the one or more alterations comprises substitution of a promoter having an altered rate of expression than the native promoter of the gene; wherein said the promoter is an inducible promoter, wherein such that the gene is expressed is expressed at altered levels compared to the baseline levels or by altered copy number of said gene, wherein the animal exhibits schizophrenic or psychotic behavior.

In some embodiments, the above transgenic nonhuman animals are used to screen for therapeutic agents that modulate symptoms of schizophrenia by administering a candidate therapeutic active agent to the transgenic nonhuman animals and determining the effect of a therapeutic active agent on symptoms associated with schizophrenia. In some embodiments of the above aspects, the transgenic nonhuman animal is a mammal. According to some aspects, the invention is based on the mapping of the mRNA expression level of proteins of the endocannabinoid system, of TNF-α and of IL-6 pro-inflammatory cytokines in a mouse model of schizophrenia (see Example 1). In particular, mRNA expression level of the two cannabinoid receptors, anabolic enzymes and the catabolic enzymes for endocannabinoids anandamide and 2-arachidonoylglycerol were examined. In addition to the effect of phencyclidine (PCP) on gene expression, the effect of BCP, a selective cannabinoid receptor type 2 (CB2) agonist which inhibits the secretion of selected pro-inflammatory cytokines, was examined in the same model.

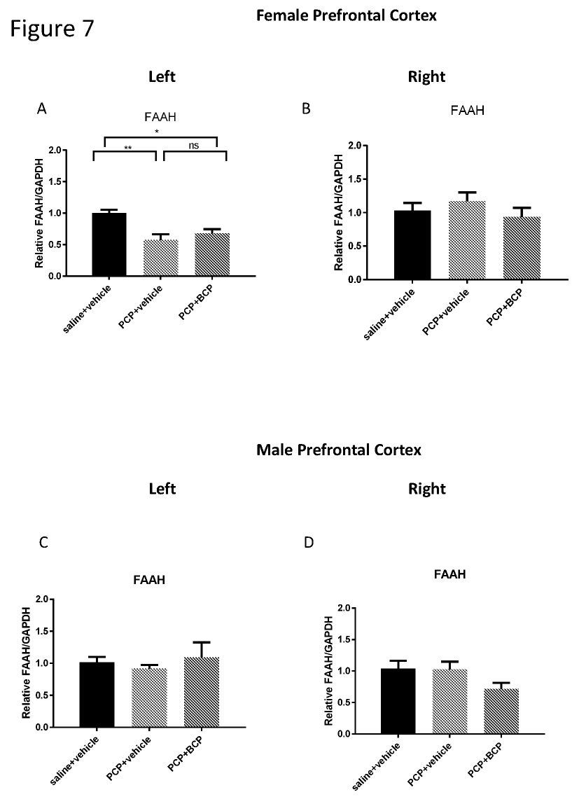

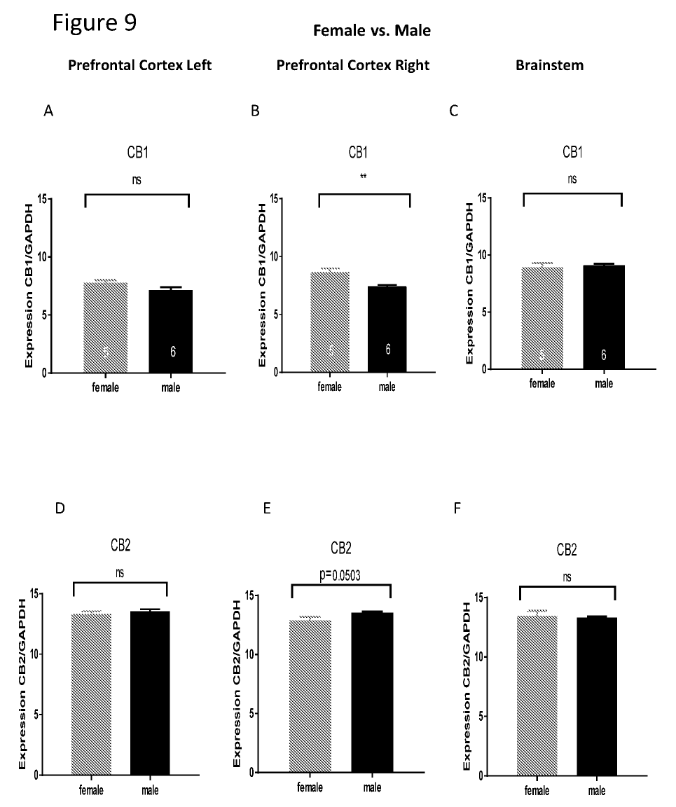

In some aspects, the mRNA expression levels of these proteins in the right and left hemisphere of prefrontal cortex of the brain were compared (see par. [0061]-[0064] and FIGS. 7A-7I, 9A-9C and 9D-9F of co-pending US patent application No. US2015051299). Schizophrenia-like symptoms were induced in postnatal mice by sub-chronic injections of phencyclidine. Phencyclidine (PCP), an N-Methyl-D-aspartate (NMDA) receptor antagonist, was selected because it induces psychosis in humans and used as drug of abuse and because it is widely used in laboratory animals to induce acute or chronic schizophrenia-like symptoms. PCP is known under the street name ‘angel-dust’.

In some aspects, the invention is focused around modulating the expression level of the targeted genes. In some aspects, the invention is focused on three brain areas, the left and right hemispheres of the prefrontal cortex and the brainstem. In the first stage of the study, the system was calibrated and the effectiveness of the PCR and RT-PCR (Real-time polymerase chain reaction) was examined. This calibration enabled to compare the mRNA expression level of selected genes at maximum efficiency. The selected genes were to the following proteins: GAD67, TNF-α, IL-6 that are related to the immune system and the cannabinoid receptors, GPR55 receptor, and the enzymes fatty acid amide hydrolase (FAAH), monoacylglycerol lipase (MGL), α/β-hydrolase domain containing 6 (ABHD6 or ABDH6) and NAPE-PLD that are related to the endocannabinoid system.

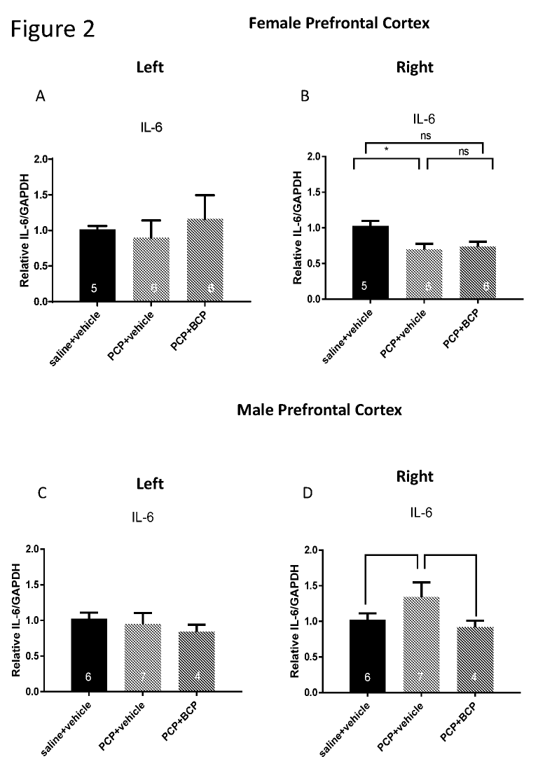

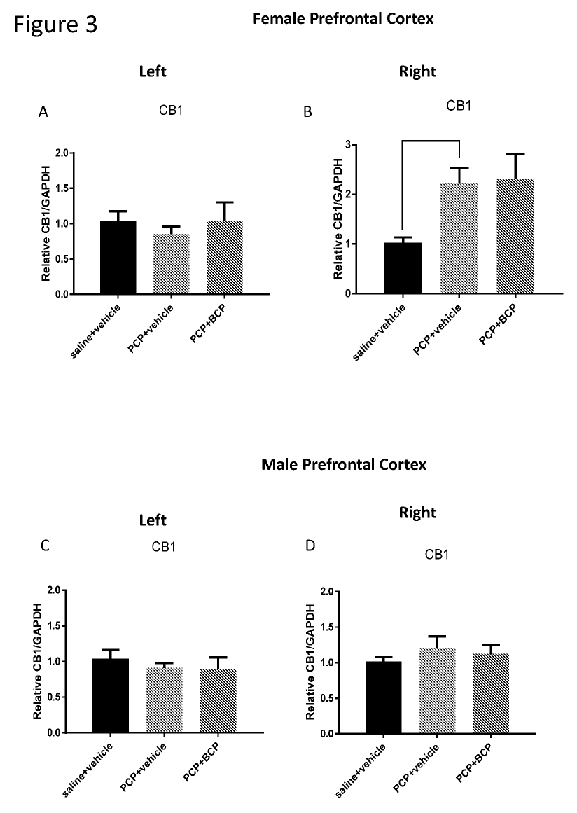

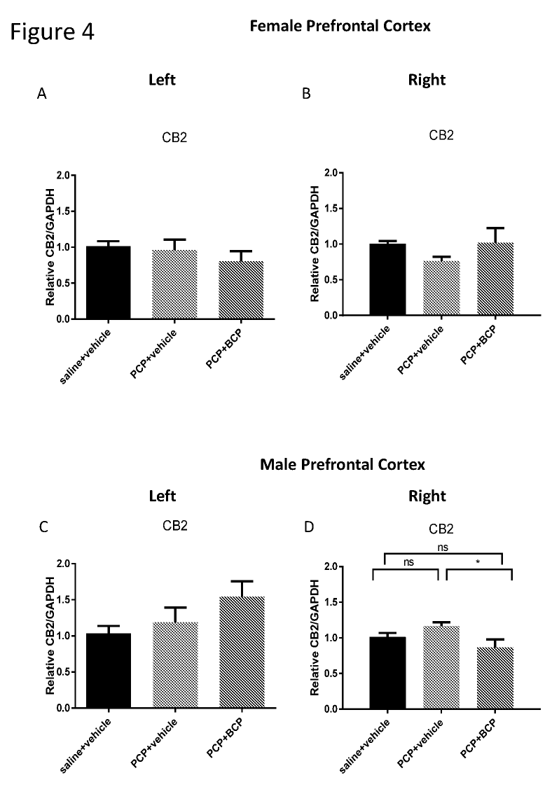

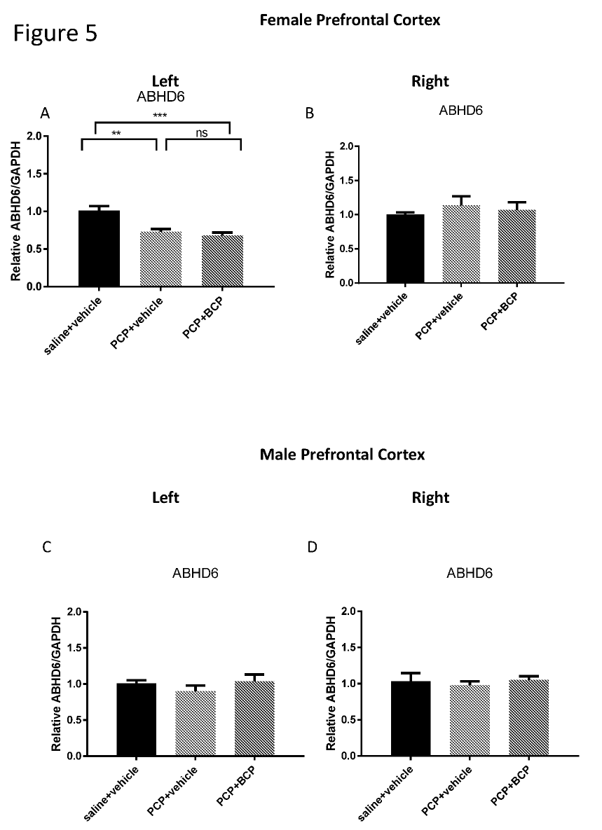

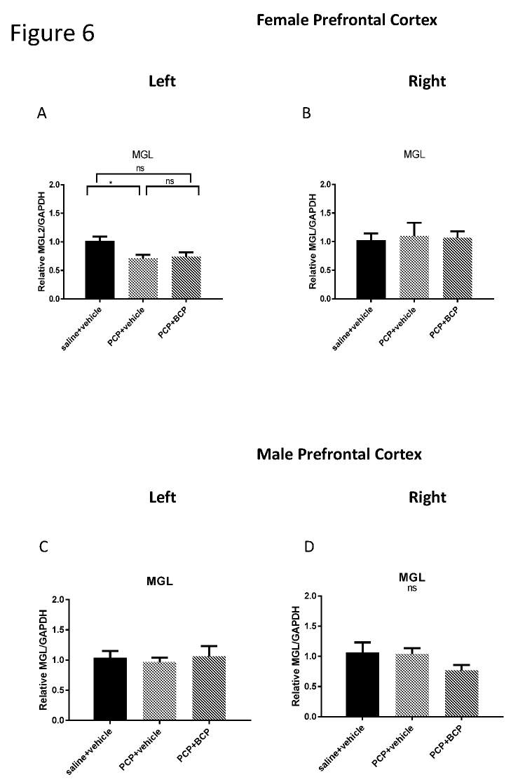

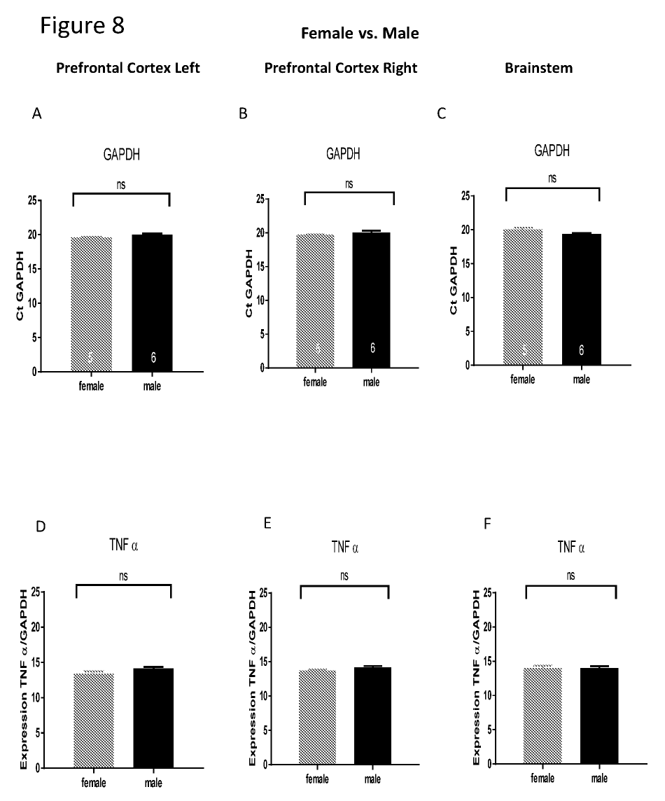

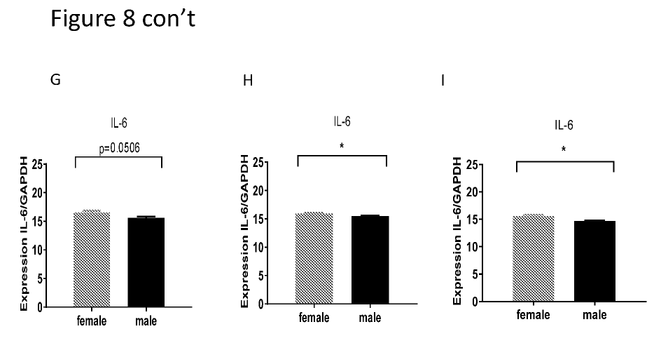

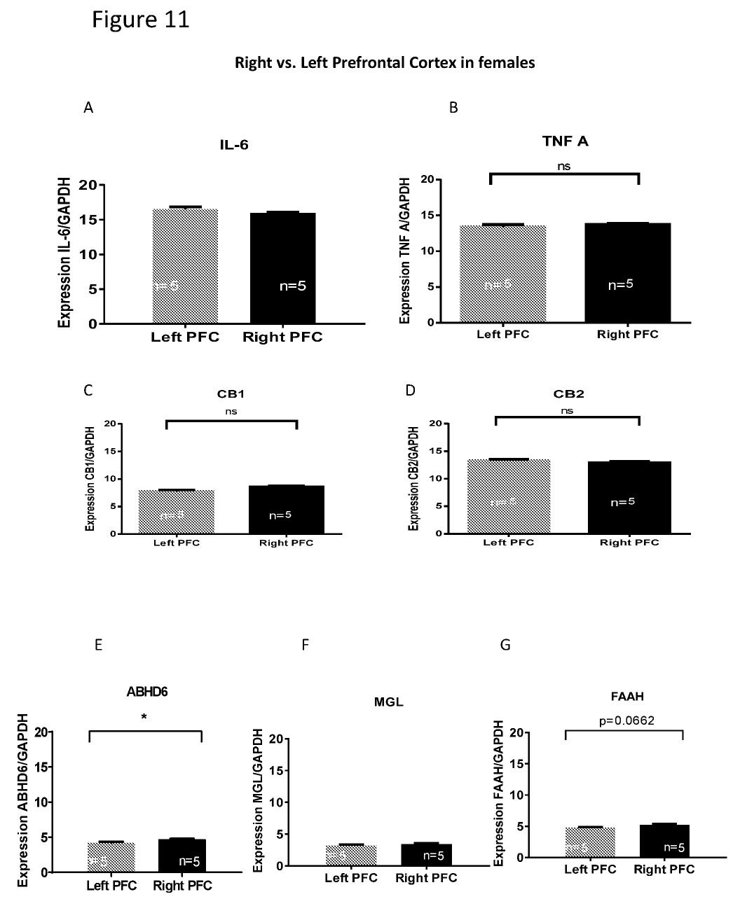

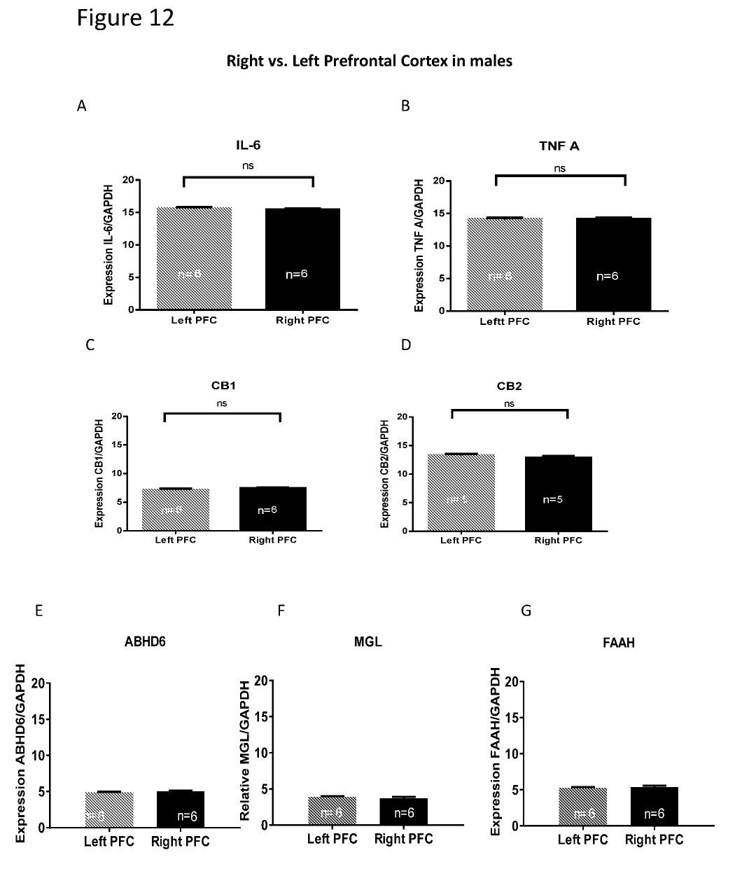

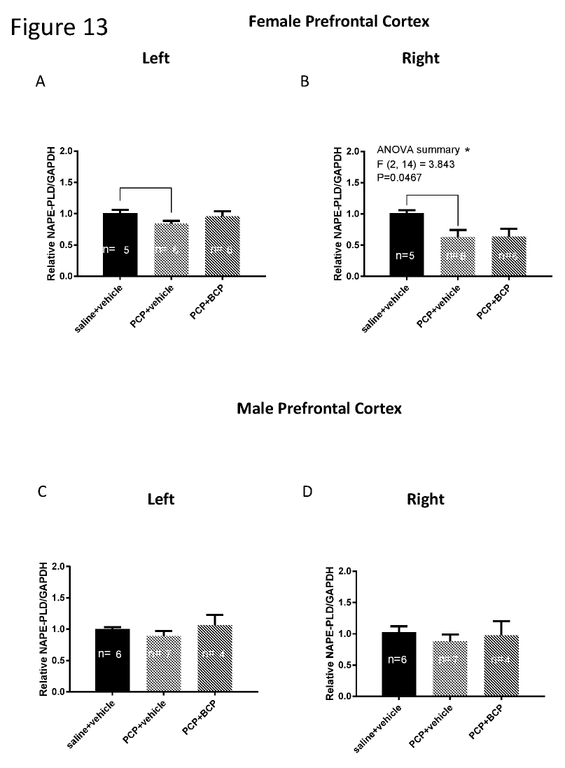

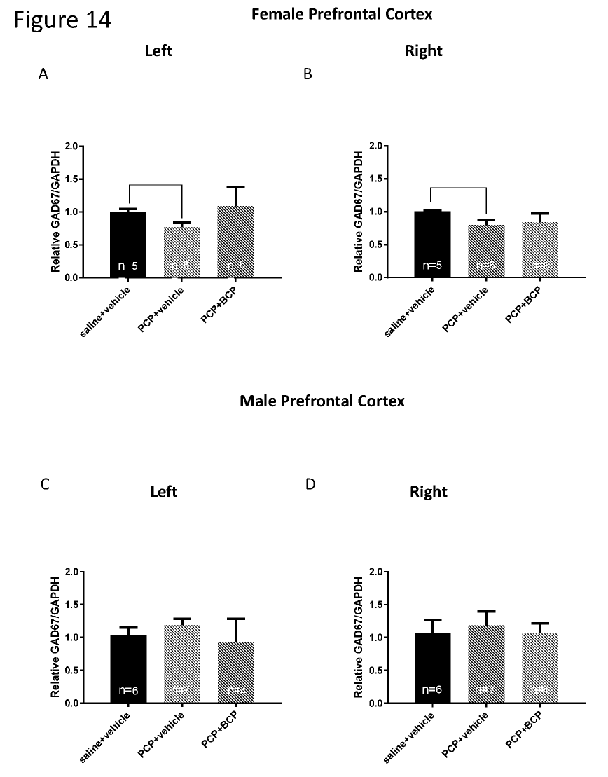

The experimental results (Example 1, FIGS. 2-14) revealed lateralization of mRNA expression in the left and right hemispheres of the prefrontal cortex. Lateralization was also found within the control group of mice. For example, in female there is a difference between the left and right hemispheres in the expression of FAAH and ABHD6 (FIG. 11). In mice, the delta Ct of mRNA level of both genes is lower in the left compared with the right hemisphere, pointing that the expression level of both genes is higher in the left than in the right (FIG. 11). Lateralization was also found in the response to PCP. Particularly interesting were the changes found in response to PCP in the right hemisphere, but not in the left hemisphere, of both males and females, in the expression level of IL-6, but not in the level of TNF-α. In females, lateralization also was found in the mRNA expression level of the endocannabinoid system; the level of the catabolic enzymes (FAAH, MGL, ABHD6) was significantly decreased in the left hemisphere of the prefrontal cortex (FIGS. 5, 6, 7, respectively) while the expression level of CB1 receptor increased in the right cortex (FIG. 3). Importantly, the results showing that expression level of the catabolic enzymes (FAAH, MGL, ABHD6) was decreased were surprising results. As previous applications e.g. U.S. Ser. No. 14/941,821, PCT/PT2014/000049 teach enzyme inhibitors for the treatment of schizophrenia, suggesting that the enzyme level is too high and there is a need to decrease its activity. Whereas the results herein suggest that the level of these enzymes is too low. Therefore, the results presented here point that a therapeutic agent/compound which enhances enzyme activity or a gene enhancer to increase protein expression of FAAH, MGL, ABHD6 is actually required for the treatment of schizophrenia, psychosis or PCP abuse or addiction.

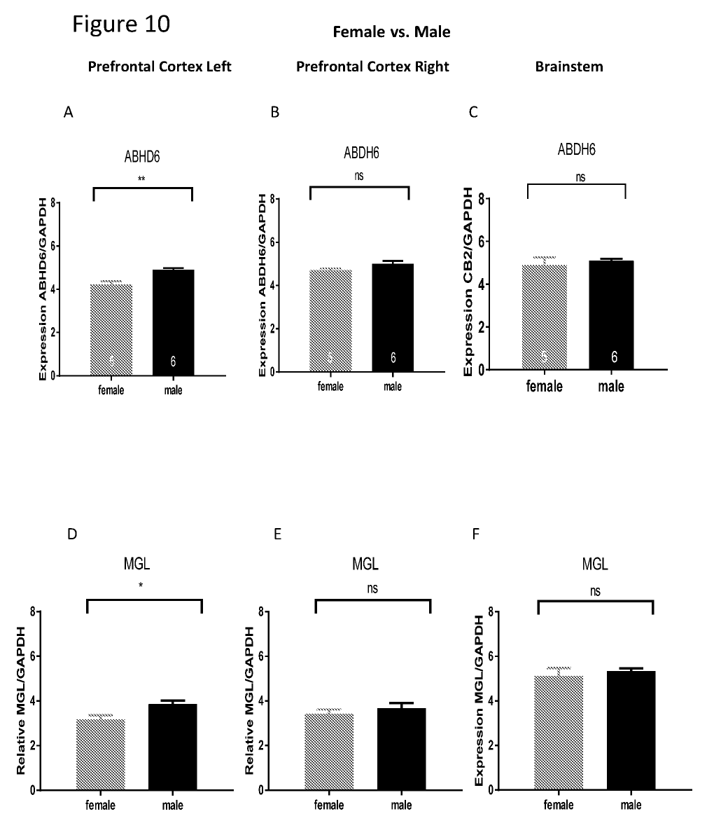

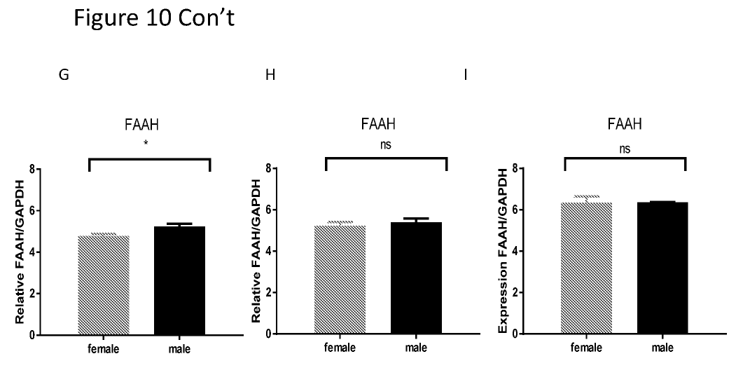

Comparison of the selected proteins between males and females revealed significant differences in mRNA expression level in the three selected brain regions. Specifically, the delta Ct of mRNA expression level of IL-6 was higher in females than in males in the right and left hemispheres of the prefrontal cortex and in the brain stem. These results suggest that IL-6 protein expression in females is lower than in males. Significantly, the delta Ct of mRNA expression level of ABHD6, MGL, FAAH (FIG. 10) was higher in males than in females in the left hemisphere, pointing that the expression level of these genes is lower in the male left hemisphere. Furthermore, in the right hemisphere, differences were found in delta Ct of mRNA expression level of the cannabinoid receptors; accordingly the level of the CB1 cannabinoid receptor is lower and the level of the CB2 cannabinoid receptor is higher in females compared to males. Collectively, these results point that the doses of therapeutic agents that modulate these genes and proteins are needed to be adjusted according to sex or their expression level.

Another important finding is that in females, treatment with BCP after the exposure to phencyclidine did not lead to significant changes in the mRNA level of the selected endocannabinoid catabolic enzymes in the cortex and brainstem. However, in the males, BCP induced changes in mRNA level and reversed the effect of PCP on ABHD6 and FAAH.

The findings of lateralization in the endocannabinoid system highlight novel approaches for treatment and diagnosis of brain diseases e.g. schizophrenia, psychosis and PCP abuse or addiction.

The findings of sex differences in the endocannabinoid system and IL-6 highlight that novel approaches for treatment and diagnosis of mental diseases, e.g. schizophrenia, psychosis and PCP abuse or addiction, should be sex-dependent.

The findings that treatment with BCP (a receptor ligand) also affects the gene level highlight novel approaches for treatment and diagnosis of brain diseases e.g. schizophrenia, psychosis and PCP abuse or addiction.

This invention contributes to our understanding of the involvement of endocannabinoid system in the pathophysiology of schizophrenia, psychosis and PCP abuse or addiction.

Similar changes were found in the expression of GAD67, IL-6 and TNF-α in a postnatal mouse model of schizophrenia to those found in adult patients with schizophrenia. The results of this study also support clinical studies that demonstrate differences between sexes and indicate an increased sensitivity to changes in endocannabinoid system in females. As CB2 receptor selective agonists lack psychoactive effects, which are attributed mainly to the stimulation of the CB1 receptor, these ligands can be potentially developed to treat mental disorders.

In some embodiments, there are provided methods of screening for mental diseases like schizophrenia, psychosis or phencyclidine (PCP) abuse or addiction in a subject or a subject population, diagnosing mental disease in a subject or in a subject population by determining the magnitude of expression of at least one gene and providing a treatment of a mental disease like schizophrenia, psychosis or PCP abuse or addiction, based on said screening and diagnosis.

Differences between the expression level of genes in the left vs. right hemispheres (lateralization) were found.

Based on the lateralization discovery of this invention, women and men can receive treatment with different therapeutic agents or their combinations. As such, drug treatment can be useful targeting specific brain region, tailored to gender and targeting a specific hemisphere.

According to aspects of the invention, for individual therapy, patients who have mutations in one of the relevant genes shall not receive specific treatments, i.e. depending on the symptoms.

Thus, for example, the active agent beta-caryophyllene (BCP) studied in this invention is recommended only for subjects lacking a mutation which result in a total inactive CB1 receptor or CB2 receptor protein.

In some embodiments, there is provided a therapeutic composition comprising BCP and a pharmaceutically effective carrier for use in screening, diagnosing, selecting and treating mental diseases selected from schizophrenia, psychosis and PCP abuse or addiction in a subject or a subject population screened, diagnosed and treated according to aspects of the invention.

Other active agents which can be used according to this invention include, but are not limited to, ‘antisense’ nucleic acids, gene enhancers, non-coding RNA transcripts, ribozymes, small molecule promoter modulators, ligands to their receptors, CB2 receptor selective agonists, CB1 receptor selective antagonists, GPR55 antagonists, BCP, HU-308, antibodies and combination thereof.

The present invention uncovered that, out of the selected genes above, some genes are upregulated in schizophrenic-like mice while others were found to be downregulated compared to baseline or normal levels.

The terms “normal” and “baseline” are used interchangeably herein. The terms “abnormal” or “different” or “departure from” are used interchangeably herein. Baseline levels are defined using conventional statistical techniques in connection with an analysis of mice which received only vehicle (saline with or without DMSO and Cremophor) but did not receive phencyclidine.

In one aspect, a method of screening for schizophrenia in a subject or a subject population is provided which includes determining, in members of the population, the magnitude of expression of a gene selected from the group consisting of the gene encoding, GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR in a sample and comparing the magnitude of expression to a baseline magnitude of expression of the gene, wherein abnormal gene expression, i.e. different from baseline of the population or the subject, indicates the presence of schizophrenia.

In another aspect, a method for diagnosing schizophrenia in a subject or in a subject population is provided, which includes determining the magnitude of expression of a gene selected from the group consisting of the gene encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR in a sample and comparing the magnitude of expression to a baseline magnitude of expression of the gene, wherein abnormal gene expression indicates the presence of schizophrenia.

In some embodiments, the sample for the above screening or diagnosing aspects, may be taken, for example, from the body fluids such as from blood, saliva, lymphatic fluid, urine or feces. In some embodiments, the sample can be taken from a body organ such as epithelial cells, spleen, skin, hair. In some embodiments, the sample can be taken from a specific left-right side of the brain, prefrontal cortex, brain stem, hippocampus and/or spinal cord.

In some embodiments, the subject is human. In other embodiments, the subject is nonhuman. In other embodiments, the population is human. In other embodiments, the population is nonhuman.

There are numerous techniques known to those with skill in the art to measure gene expression in a sample. For example, DNA, RNA from a cell type or tissue known, or suspected, to express the genes GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR, such as brain, may be isolated and tested utilizing hybridization or PCR, RT-PCR techniques such as are described above and below. The isolated RNA can be derived from a cell prepared by a primary culture or differentiated lineage from a patient or directly from a biological sample from a subject.

This invention reveals novel aspects related to schizophrenia, psychosis and PCP abuse or addiction, allowing new methods for the screening, diagnosis and treatment of these medical conditions, as detailed below.

This invention discovered lateralization in the expression of targeted genes related to the endocannabinoid system as shown in a mouse model for schizophrenia. The targeted genes are expressed at different levels in the right hemisphere vs. left hemisphere.

Alterations in IL-6 mRNA expression in the model described herein are similar to those found in schizophrenic subjects. This suggests that the conclusions resulting from the findings of lateralization in the endocannabinoid system and other target genes apply to schizophrenic subjects.

Therefore, these novel findings can be used for targeted treatments by directing gene therapy to specific hemisphere and for diagnosis of schizophrenic, psychotic or PCP abused or addicted subjects.

The expression of the targeted genes is gender-specific (different between males and females). Especially in the female, the delta Ct level of the mRNA of the enzymes ABHD6, FAAH and MGL is significantly decreased in left cortex of mice in the PCP-schizophrenia model, suggesting a higher level of ABHD6, FAAH and MGL enzymes in the females than in males. Importantly, BCP did not reverse in females the effect of PCP on the expression level of these genes, pointing that a combination of therapeutic agents is required for novel treatment of mental disease.

These findings point out to the facts that:
(1) The treatment needs to be adjusted according to gender;
(2) Treatment of females with BCP alone may not be enough;
(3) Within the same model of schizophrenia, other members of the endocannabinoid system are affected, for example the expression of the mRNA level of selected catabolic enzymes is decreased.

The above results are surprising and totally unexpected. BCP, a ligand to the CB2 receptor, was found to modulate the expression level of other targeted genes related to the endocannabinoid system and to the immune system, and not only those of the CB2 receptor.

These novel findings highlight the fact that the treatment with BCP also affects at the gene level.

Other surprising results were related to the decreased enzyme level in the model of schizophrenia. These findings highlight that ABHD6, FAAH, MGL enzyme enhancers or gene enhancers are required for the treatment of mental diseases.

The methods of treatment detailed in this disclosure, use an active agent selected from the group consisting of “antisense” nucleic acids, gene enhancers, non-coding RNA transcripts, ribozymes, small molecule promoter modulators, CB2 receptor selective agonists, CB1 receptor selective antagonists, GPR55 antagonists, BCP, HU-308, rosmarinic acid, an antibody (whole antibody, humanized antibody, chimeric antibody, Fab fragment, Fab′ fragment, F(ab′)2 fragment, single chain Fv fragment and diabody) and combinations thereof.

The above methods of treatment are preceded by a screening and diagnostic stage needed for the screening (selection and identification) of subjects susceptible to be responsive to the treatment in general on the one hand and on the other hand to which active agent(s) in particular.

### Diagnosis and Screening of Mental Diseases Based on Genetic Methods

This stage has several purposes:
1. Quantifying and/or visualizing the level of a targeted gene/protein expression level by an imaging system based on an imaging technology, for example, radiography, magnetic resonance, positron emission tomography (PET), single-photon emission computed tomography (SPECT), fluorescence, ultrasonography, X-ray, magnetic particle imaging.
2. Measuring the level of targeted genes or proteins using RT-PCR, kits or arrays.
3. Screening for schizophrenia, psychosis or PCP addiction or abuse in a subject population, wherein the subject population is human or nonhuman.
4. Screening for and diagnosing of schizophrenia, psychosis or PCP addiction or abuse in a subject population comprising and determining the magnitude of expression in a subject sample of at least one gene selected from GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD and comparing the magnitude of expression to a baseline magnitude of expression of the gene, wherein abnormal altered gene expression indicates the presence of a mental disease, selected from schizophrenia, psychosis or PCP addiction or abuse in a said subject.
5. Diagnosing schizophrenia, psychosis or PCP addiction or abuse in a subject as detailed above, wherein the subject sample is harvested from the body fluids selected from blood, saliva, lymphatic fluid, urine or feces of said subject.
6. Diagnosing schizophrenia, psychosis or PCP addiction or abuse in a subject as detailed above, wherein the subject sample is harvested from body organs selected from epithelial cells, spleen, skin, hair of said subject.
7. Diagnosing schizophrenia, psychosis or PCP addiction or abuse in a subject as detailed above, wherein the subject sample is harvested from the left or right side of the brain, prefrontal cortex, brain stem, hippocampus and/or spinal cord of said subject.

Some embodiments of this invention provide compositions comprising a CB2 receptor agonist selected from beta-caryophyllene (BCP), HU-308, and rosmarinic acid and combinations thereof.

In some embodiments, there are provided methods for preparing the above compositions.

In some embodiments, there are provided methods of treatment using a therapeutically effective dose of a CB2 receptor agonist for the treatment of mental diseases selected from schizophrenia, psychosis and PCP-addiction.

In some embodiments, there is provided a kit comprising a CB2 receptor agonist active agent selected from BCP, HU-308 and combinations thereof for the treatment selection, diagnosis or combinations thereof of a mental disease selected from schizophrenia, psychosis and PCP abuse or addiction.

Some embodiments of the invention relate to compositions comprising Cannabinoid Receptor Type 2 (CB2) receptor agonists, methods of making the compositions, methods for the treatment of a mental disease schizophrenia by genetic methods using CB2 receptor agonists and a kit comprising CB2 receptor agonists for the treatment and diagnosis of schizophrenia.

According to some embodiments of the invention, subjects that have mutations which abolish protein function in one of the listed above genes should not receive a specific treatment. For example, depending on the symptoms, beta-caryophyllene (BCP) should not be recommended for treatment of a subject having a mutation in the CB1 receptor or CB2 receptor gene which leads to absolute inactive CB1 receptor or CB2 receptor protein.

Likewise, women and men should receive different therapeutic agents or their combinations.

According to some embodiments of the invention, there is provided a therapeutic composition comprising a therapeutically effective dose of BCP and a pharmaceutically effective carrier for use in treating a mental disease like schizophrenia, psychosis or PCP addiction and abuse. In some embodiments, the composition is used for the treatment of a human subject. In some embodiments, the composition is used in the treatment of a non-human subject.

In some embodiments, wherein the mental disease is schizophrenia, the schizophrenia is selected from the group consisting of paranoid schizophrenia, disorganized schizophrenia, undifferentiated schizophrenia, catatonic schizophrenia and residual schizophrenia.

In some embodiments, the mental disease is psychosis, schizophrenia of all symptoms and onset at any age.

In some embodiments, the schizophrenia treatment comprises treating at least one symptom of schizophrenia selected from the group consisting of a negative symptom of schizophrenia and a positive symptom of schizophrenia. In some embodiments, the treatment of schizophrenia, psychosis or PCP abuse or addiction comprises treating any symptoms of the mental disease. In some embodiments, the PCP addiction or abuse is selected from the group consisting of short or long term abuse.

In some embodiments, the positive symptoms of schizophrenia can include one or more of psychosis, delusions, hallucinations, conceptual disorganization, excitement, grandiosity, suspiciousness/persecution, hostility, disorganized thoughts and unpredictable actions, and change in appearance or dress.

In some embodiments, the negative symptoms of schizophrenia can include one or more of blunted affect, emotional withdrawal, poor rapport, passive/apathetic social withdrawal, difficulty in abstract thinking, lack of spontaneity and flow of conversation, stereotyped thinking, inability to express emotions (‘flat effect’ or emotional flatness), lack of concentration, lack of motivation, change in sleeping and sex patterns, lack of speech, anhedonia, reduced social contact, anxiety and depression.

In some embodiments, the cognitive symptoms of the mental disease can include one or more of inability to process information, impaired decision making abilities, reduced ability to pay attention, and impaired memory.

In some embodiments, depression and anxiety and impaired memory can accompany the mental disease, schizophrenia psychosis, PCP abuse and addiction.

In some embodiments, the treatment for PCP abuse and addiction comprises treating at least one symptom of PCP abuse or addiction selected from sedation, immobility, amnesia, numbness, speech difficulties, a sense of invulnerability, blank stare, rapid, involuntary eye movements, hallucinations, high blood pressure, rapid heartbeat. Other symptoms that may show up when PCP is used over a long period of time are selected from stuttering, impaired memory, inability to think clearly, inability to speak, suicidal thoughts, anxiety and depression.

In some embodiments, the pharmaceutically effective carrier comprises dimethyl sulfoxide (DMSO). In some such embodiments, the pharmaceutically effective carrier comprises DMSO, saline and Cremophor EL. In some embodiments, the pharmaceutically effective carrier comprises DMSO, saline and Cremophor EL at a ratio of 1:0.6:18.4 Cremophor EL: DMSO: saline.

In some embodiments, the pharmaceutically effective carrier comprises DMSO, saline and Cremophor EL at a ratio of 1:1:18.4 Cremophor EL: DMSO: saline.

In some embodiments, the pharmaceutically effective carrier comprises ethanol (EtOH). In some embodiments, the pharmaceutically effective carrier comprises EtOH, saline and Cremophor EL. In some embodiments, the pharmaceutically effective carrier comprises DMSO, saline and Cremophor EL at a ratio of 1:0.6:18.4 Cremophor EL: EtOH: saline.

In some embodiments, the pharmaceutically effective carrier comprises DMSO, saline and Cremophor EL at a ratio of 1:1:18.4 Cremophor E: EtOH: saline.

In some embodiments, the pharmaceutically effective carrier comprises a self-emulsifying composition, as exemplified in co-pending International application No. PCT/US2017/20639 and in U.S. patent application Ser. No. 62/303,508, which are incorporated herein in their entireties.

### Animal Model of Schizophrenia

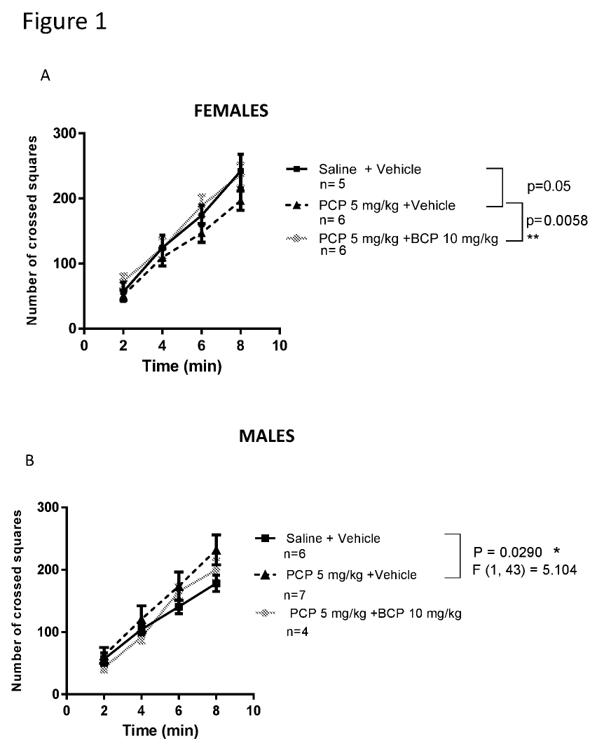

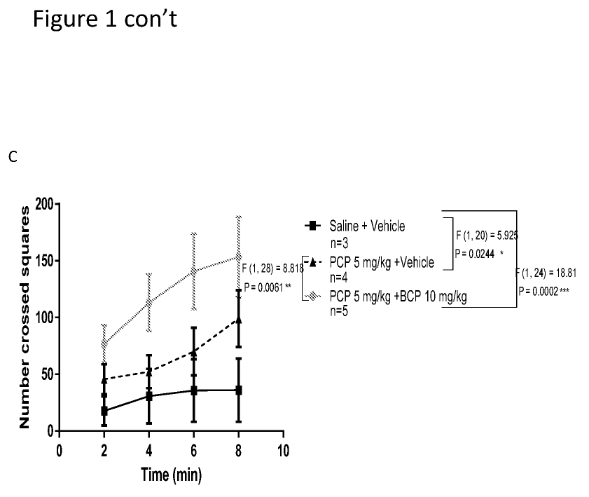

The mouse model of schizophrenia was established (Example 1 and FIG. 1). Phencyclidine (PCP), an NMDA antagonist which induces schizophrenia and psychotic effects in humans, was administered to murine pups (injection of 5 mg/kg in saline) on postnatal days 3, 6, 8, 10, 13, 16, and 18 (or 3 times a week, on alternate days, for 2.5 weeks). This treatment induces long-lasting schizophrenic-like effects in mice that last into adulthood. The therapeutic effects of beta-caryophyllene, a dietary cannabinoid and CB2 receptor selective agonist, were evaluated in accordance with the teachings herein.

The methods provided herein enable screening therapeutic agents which modulate the relevant genes in a subject in need thereof.

The screening for therapeutic agents able to modulate relevant genes is done using
1. A homozygous knockout animal—for individual or combined genes;
2. A heterozygous knockout animal—for individual or combined genes, while screening candidate active agents;
3. An overexpressing animal—for individual or combined genes while screening candidate active agents;
4. A conditional mutant animal—knockout/overexpressing in specific brain area—for individual or combined genes.
5. A mutant animal tailored to a gene mutation of a selected patient/subject, stably integrated in its genome or a conditional/site-directed mutation/tissue-specific mutation- for individual or combined genes.

The methods described herein enable screening subject populations for susceptibility to mental diseases. Populations can be screened and diagnosed for subject susceptibility to disease, for example by using gene, protein arrays.

Personalized medical treatment can be provided by screening candidate active agents for individual subjects by:
1. Carrying out genetic tests and diagnosing the effect of published or unpublished mutations.
2. Genetic/functional tests of Cytochrome P450 enzymes (CYPs) may be carried out for determining best active agent(s) or combinations thereof.
3. Diagnosing the effects of an active agent in a subject comprising determining the magnitude of expression of at least one gene selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD or 5-HT receptors in a sample and comparing the magnitude of expression to a baseline magnitude of expression of the gene, wherein alteration of the gene expression indicates that the therapeutic active agent has an effect.

In some embodiments, the therapeutic active agents screening is carried out by conducting personalized in vitro/cell culture response screening for therapeutic active agents.

In some embodiments, there is provided a method of treatment and diagnosis of a subject in need thereof, based on changes in combinations of the disclosed targeted genes.

In some embodiments, there is provided a method of treatment of schizophrenia, psychosis or PCP abuse or addiction, wherein the method is gender-adjusted, based on differences between females and males and adjusted according to gender.

In some embodiments, there is provided a method of genetic treatment of a subject in need thereof, by antisense, ribozymes (block synthesis) or by RNA enhancers (enhance synthesis). Genes having their expression reduced by PCP can be enhanced by RNA enhancers and genes having their expression enhanced by PCP can be reduced by antisense/ribozymes to block synthesis or their respective-enhancers can be silenced.

In some embodiments, there is provided a method of treatment of a mental disease subject in need thereof with a gene modulator or a cocktail of gene modulators.

In some embodiments, the specific brain areas targeted are mainly the cortex and the brain stem, spinal cord, but not exclusively.

In some embodiments, there is provided a method of treatment of a mental disease subject in need thereof with enhancers/antisense or other inhibitors to CB2 receptor mRNA.

In some embodiments, there is provided a method of treatment of a mental disease subject in need thereof with enhancers/antisense or other inhibitors with or without another therapeutic agent e.g. drug therapy.

In some embodiments, there is provided a method of treatment of a mental disease subject in need thereof based on the modulation of targeted molecules/sequences controlling the degradation of endogenous enhancers to genes of the endocannabinoid system.

In some embodiments, there is provided a transgenic nonhuman knockout animal whose genome comprises a homozygous disruption in one or more genes selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combination thereof, wherein said homozygous disruption prevents the expression of the gene, and wherein said homozygous disruption results in a transgenic knockout animal exhibiting decreased expression levels of the one or more genes as compared to a wild-type animal.

In some embodiments, there is provided the above transgenic nonhuman animal, wherein the transgenic nonhuman animal is a mammal.

In some embodiments, there is provided a method for screening and treatment of a mental disease selected from schizophrenia, psychosis and PCP addiction or abuse in a subject or a subject population, the method comprising:
a. Screening a subject or a subject population for genetic mental disease susceptibility;
b. Diagnosing in a subject or subject population the mental disease susceptibility and the at least one gene in a subject sample causing the mental disease susceptibility;
c. Determining in a subject or subject population whether at least one of the genes causing the mental disease susceptibility has a gene mutation;
d. Selecting out of a group of candidate active agents at least one active agent exhibiting activity in altering the expression of the at least one gene causing the mental disease susceptibility;
e. Treating the mental disease subject with a therapeutically effective amount of the at least one active agent selected out of the group of candidate active agents or combinations thereof;

wherein the above treatment with a therapeutically effective amount of at least one active agent is gender-specific based on lateralization findings, and

wherein a subject having a gene mutation causing protein total inactivation in at least one of the genes leading to the mental disease susceptibility is not treated with the selected active agent.

In some embodiments, there is provided the above method, wherein screening a subject or a subject population for genetic mental disease susceptibility comprises determining in a subject sample the magnitude of expression of at least one gene causing the mental disease susceptibility in a subject or a subject population, and comparing this magnitude of expression to a baseline magnitude of expression of the at least one gene, wherein departure from baseline magnitude of expression of at least one gene indicates the presence of a mental disease selected from schizophrenia, psychosis or PCP addiction or abuse.

In some embodiments, the at least one gene in a subject sample causing the mental disease susceptibility is selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptor and combinations thereof.

In some embodiments, the above subject sample is harvested from a body fluid selected from cerebrospinal fluid (CSF), blood, saliva, lymphatic fluid, urine and feces, or from a body organ selected from epithelial cells, spleen, skin, and hair or from a specific left or right side of the brain, prefrontal cortex, brain stem, hippocampus and/or spinal cord of a human or a nonhuman subject.

In another embodiment, the diagnosing of the mental disease susceptibility in a subject and the specific at least one gene causing the genetic susceptibility comprises screening, quantifying, visualizing, measuring the expression level and detecting departures from baseline of at least one gene selected from genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof, using gene sequencing, PCR, RT-PCR, imaging systems, kits, arrays targeting DNA, RNA, protein in whole body or of a cell or a tissue sample harvested from a human or a nonhuman subject.

In some embodiments, the method comprises determining in a subject or subject population whether the subject or subject population has a gene mutation leading to the mental disease susceptibility, comprises comparing in a subject sample the magnitude of expression of at least one gene causing the mental disease susceptibility and comparing the magnitude of expression to a baseline magnitude of expression of the gene, wherein altered gene expression indicates the presence of a mental disease, selected from schizophrenia, psychosis or PCP addiction or abuse.

In some embodiments, the method comprises selecting out of a group of candidate active agents at least one active agent exhibiting activity in altering the magnitude of expression of the at least one gene causing the mental disease susceptibility selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof, wherein the selected active agent improves any symptoms of the mental disease, selected from schizophrenia, psychosis or PCP addiction or abuse in a human or a nonhuman subject.

In some other embodiments, the method of treating a mental disease in a subject or subject population comprises administering to a subject in need thereof a therapeutically effective amount of at least one selected active agent or combinations thereof, wherein the group of candidate active agents consists of a gene inhibitor selected from an antisense oligonucleotide, a nucleic acid molecule, an interfering RNAs (RNAi) selected from a small interfering RNA (siRNA), a micro interfering RNA (miRNA), an RNA-induced transcriptional silencing (RITS), a ribozyme and combinations thereof, a gene enhancer selected from, a short DNA enhancer, an eRNA enhancer molecule and combinations thereof, a gene modulator selected from non-coding RNA transcripts, small molecule promoter modulators and combinations thereof, a CB2 selective agonist selected from BCP, HU-308, a FAAH enhancer, a MGL enhancer, rosmarinic acid and combinations thereof, an antibody selected from whole antibody, humanized antibody, chimeric antibody, Fab fragment, Fab′ fragment, F(ab′)2 fragment, single chain Fv fragment, diabody and combinations thereof.

In some embodiments, the above method of treatment comprises administering to a subject in need thereof a therapeutically effective amount of at least one selected active agent altering the magnitude of expression of at least one gene selected from the group consisting of GAD1, IL6, TNF, CNR1, CNR2, GPR55, FAAH, MAGLL, ABHD6, ABHD12, ABHD4, DAGLA, DAGLB, NAPEPLD, GDE1, PLD, PLC, HTR and combinations thereof, wherein the active agent is selected from
a. A gene expression lowering amount of an antisense oligonucleotide, a siRNA, a ribozyme, a nucleic acid molecule or combinations thereof;
b. A gene expression enhancing amount of a gene enhancer, a nucleic acid molecule or combinations thereof;
c. A gene expression altering amount of at least one RNAi molecule or combinations thereof;
d. A gene expression altering amount of at least one a gene enhancer molecule or combinations thereof;
e. A gene expression enhancing amount of at least one non-coding RNA transcript or combinations thereof;
f. A gene expression altering amount of at least one RNA-cleaving ribozyme RNA or combinations thereof;
g. A gene expression altering amount of at least one small molecule promoter modulator or combinations thereof;
h. A gene expression altering amount of at least one CB2 selective agonist or combinations thereof;
i. A gene expression altering amount of BCP;
j. A gene expression altering amount of HU-308;
k. A gene expression altering amount of ABHD6 enhancer;
l. A gene expression altering amount of MGL enhancer;
m. A gene expression altering amount of FAAH enhancer;
n. A gene expression altering amount of rosmarinic acid;
o. A gene expression altering amount of an antibody selected from the group consisting of whole antibody, humanized antibody, chimeric antibody, Fab fragment, Fab′ fragment, F(ab′)2 fragment, single chain Fv fragment, diabody and combinations thereof
p. Any combinations of the above (a)-(o) active agents.

In another embodiment, there is provided the above method of treatment with an antibody, wherein the said antibody specifically binds to an epitope of IL-6, TNF-alpha, CB1 receptor, CB2 receptor, GPR55, a 5-HT receptor or combination thereof prior to the manufacture of a medicament for the treatment of a mental disease selected from schizophrenia, psychosis and PCP abuse or addiction.

In some embodiments, there is provided a method of treatment of a mental disease selected from schizophrenia, psychosis and PCP abuse or addiction in a subject or subject population, wherein comprising reducing/increasing/stabilizing the amount of at least one protein encoded by at least one of the genes selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof by administration of a therapeutically effective amount of antibody or functional antibody fragment.

In other embodiments, there is provided a method of screening candidate active agents for the treatment in a subject in need thereof of a mental disease selected from schizophrenia, psychosis and PCP abuse or addiction, comprising administering a candidate active agent to a transgenic or a knockout or a conditional nonhuman animal and determining whether the active agent cures and/or improves at least one of the schizophrenia, psychosis or PCP abuse or addiction symptoms.

In some embodiments, there is provided a transgenic, knockout or conditional nonhuman animal comprising stably integrated in its genome a gene selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof, wherein expression of the gene is altered by one or more changes in the regulatory sequences/conditional controlling mechanisms of the gene such that the gene is expressed at altered levels compared to the baseline levels or by altered copy number of said gene, wherein the animal exhibits schizophrenic or psychotic behavior.

In some other embodiments there is provided a nonhuman animal comprising a gene mutation of a selected patient/subject, stably integrated in its genome or a conditional/site-directed mutation/tissue-specific mutation, selected from the group consisting of genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof, wherein expression of the combination of the genes is decreased/enhanced by one or more alterations in regulatory sequences/conditional controlling mechanisms, or a regulatory sequences of the gene, wherein the one or more alterations comprises substitution of a promoter having an altered rate of expression than the native promoter of the gene, wherein the promoter is an inducible promoter, such that the gene is expressed at altered levels compared to the baseline levels or by altered copy number of said gene, and wherein the animal exhibits schizophrenic or psychotic behavior.

In some embodiments a transgenic, a knockout, a conditional nonhuman animal or an animal comprising a gene mutation of a selected patient/subject is used in methods of screening, selecting, determining of a candidate therapeutic agent.

In some embodiments, there is provided a method of screening for a candidate active agent for the treatment of a mental disease selected from schizophrenia, psychosis and PCP abuse or addiction comprising operatively linking a reporter gene which expresses a detectable protein to a regulatory sequence for a gene selected from the group consisting of genes encoding GAD67, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combinations thereof, to produce a reporter construct, transfecting a cell with the reporter construct, exposing the transfected cell to a candidate active agent and comparing the level of expression of the reporter gene after exposure to the tested compound/agent to the level of expression before exposure to the tested active agent, wherein an alteration in the level of expression after exposure is indicative of candidate active agent useful for the treatment of a mental disease.

In some other embodiments, there is provided a kit comprising a custom array selected from a gene array, a probe array, a protein array, an array comprising a therapeutic agent, a nucleic acid molecule which selectively hybridizes to a nucleic acid molecule, a cell or a kit component which expresses a patient's mutation, to at least one of the genes selected from genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, 5-HT receptors and combination thereof, and instructions for use it in a combination with other genes/proteins and combination thereof.

In some embodiments, there is provided a method of screening for a CB2 selective receptor active agent for the treatment of a mental disease selected from schizophrenia, psychosis and PCP abuse or addiction, wherein the screening is done in a native or constructed cell or in a transgenic/knockout animal expressing at least one 5-HT receptor or at least one mutant 5-HT receptor and combination thereof, by comparing the cell or animal response before and after exposure to the tested active agent, wherein an altered level of response is indicative of suitability for the treatment of schizophrenia, psychosis and PCP abuse or addiction.

In some other embodiments, there is provided a method of selection of candidate active agents and combination thereof, comprising determining the gene expression or functional activity of a Cytochrome P450 enzyme in an homogenate mix, a cell, a mutant cell, a tissue or an organ originating from a human, an animal or a transgenic, knockout or conditional animal, wherein an alteration in the magnitude of a Cytochrome P450 enzyme activity or its gene expression is indicative of the active agent suitability for the treatment of schizophrenia, psychosis and PCP-addiction.

Aspects of the invention relate to a method for screening, diagnosis or treatment, wherein the subject is human.

Aspects of the invention relate to a method for screening, diagnosis or treatment, wherein the mental disease is schizophrenia or psychosis and wherein said schizophrenia, psychosis includes any symptom and its onset is at any age.

In some embodiments, the subject is human. In other embodiments, the subject is nonhuman. In other embodiment, the population is human. In other embodiment, the population is nonhuman.

The results disclosed here further support the therapeutic effect of BCP through the CB2 receptor for the treatment of schizophrenia. See U.S. patent application Ser. No. 14/385,739 (published as US2015/051299) incorporated by reference in its entirety.

Aspects of the invention provide methods of screening for a mental disease selected from schizophrenia, psychosis and phencyclidine abuse and addiction in a subject or a subject population, diagnosing schizophrenia, psychosis and phencyclidine abuse and addiction in a subject by determining the magnitude of expression of at least one gene and providing tools for selection of a treatment and a list of therapeutic agents for the treatment of schizophrenia, psychosis and phencyclidine abuse or addiction based on said screening and diagnosis of a human or a nonhuman animal.

Differences between the expression levels of at least one in the left vs. right hemispheres (lateralization) were found.

Based on the lateralization discovery of this invention women and men can be treated with different combinations of therapeutic agents. As such, drug treatment can be be useful targeting specific brain region, a specific hemisphere and tailored to gender and age.

Other drugs that can be used according to this invention include a gene inhibitor, a gene enhancer, a gene modulator, a CB2 selective agonist selected, a FAAH enhancer, a MGL enhancer, rosmarinic acid and combinations thereof, an antibody or an antibody fragment and combination thereof.

Kits and instructions are provided, comprising a custom array or comprising a therapeutic agent and their combination thereof.

Aspects of the invention may be used for:

Genetic Counselling
1. Individuals and families concerned of being at high risk of developing schizophrenia due to multiple schizophrenia cases in the family;
2. Identification of a genetic mutation in a family member;
3. Specific populations with higher rate of schizophrenia compared to other populations (e.g. Japanese, Scottish);
4. Family history that is unclear for various reasons (such as adoption, egg/sperm donation);

Counselling Aims
1. Evaluation of the personal and familial risk level of developing schizophrenia or psychosis;
2. Designing a personal plan for early detection or risk reduction;
3. Assessing the need for genetic testing and selection of the relevant tests. The tests are performed simply and easily on the spot by taking a sample of cerebrospinal fluid (CSF), blood, saliva, lymphatic fluid, urine or feces, or from a body organ selected from epithelial cells, spleen, skin, hair;
4. For subjects with current or previous schizophrenia—better diagnosis of disease characteristics; assessment of the risk level of family members such as children, siblings, uncles/aunts;
5. Personalized medical recommendations for mental disease treatment. The subject can receive a risk assessment and personalized medical recommendations.

### EXAMPLES

The following examples illustrate certain embodiments of the invention but are not meant to limit the scope of the claims in any way. The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### PCP Induced Schizophrenia-like Symptoms and Altered the Expression Level of Genes Related to the Endocannabinoid and Immune Systems. Treatment with BCP Reversed Alteration in Gene Expression

Materials and Methods

BCP was obtained from Sigma-Aldrich (St. Louis, Mo., USA), catalogue No. W225207 and further purified using preparative HPLC (HP1090 series; column, PEGASIL ODS (Senshu Sci. i.d. 10×250 mm); solvent, 70% CH₃OH; flow rate, 2.0 mL/min; detection, UV 220 nm] to remove other sesquiterpenes. GC-MS analysis showed that the BCP used in the below Example 1-2 contained E-BCP, alpha humulene and traces of Z-BCP, BCP oxide. PCP, Cremophor EL and DMSO were obtained from Sigma-Aldrich (St. Louis, Mo., USA).

PCP (5 mg/kg; Sigma-Aldrich) or saline was injected to Sabra mice (Harlan, Israel) or CB1 receptor knockout C57Bl/6J mice. One hour later, animals were injected with BCP (final dose 10 mg/kg in 1:0.6:18.4 Cremophor EL: DMSO: saline) or vehicle (1:0.6:18.4 Cremophor EL: DMSO: saline).

Results (see FIGS. 1-14) were obtained from three different mice litters, each of which was divided according to sex into 3 groups:

Group 1: Saline+vehicle (number of animals is indicated on each graph in FIG. 1);

Group 2: Saline+PCP (number of animals is indicated on each graph in FIG. 1);

Group 3: PCP+BCP (number of animals is indicated on each graph in FIG. 1).

### Example 2

### Assessment of Positive Schizophrenic-like Behavior

Open-Field Test (Rearing)

In FIG. 1, mice were assessed for hyperactivity behavior on postnatal day 20-22 (FIG. 1). Mice were placed in the center of a transparent glass cube cage 30×40 X31 cm divided into squares of 7.5×7.5 cm. The number of squares and rearing activity were counted for 8 min with 2 min intervals.

Brain Dissection

On postnatal day 26 or 27 the mouse brains were removed and dissected in cold HEPES buffer (Coutts et al., 2002) on ice-cooled tray. Each brain area was placed in an Eppendorf vessel and covered with RNAlater® solution (Ambion). Eppendorfs were stored overnight at 4° C. and then stored at (−)80° C. for permanent use.

TABLE 1

| Product | Manufacturer | Cat. No |
|---|---|---|
| TRI reagent | Sigma-Aldrich | 93289-25ML |
| 2-Propanol | Sigma-Aldrich | I9516-25ML |
| 1-Bromo-3-chloropropane | Sigma-Aldrich | B9673-200ML |
| RNAlater | Ambion | AB-AM7021 |
| Turbo DNA-free 50 rxns/PC/ | Ambion | AB-AM1907 |
| High Capacity cDNA RT kit | Applied | AB-4374966 |
| 200Rxn + 2 × RNAse 1 inhibitor | Biosystems | |
| Power SYBR ® Green PCR Master | Applied | AB-4367659 |
| Mix, 10-Pack 5 mL | Biosystems | |

Preparing cDNA

RNA extraction was done according to manufacturer's recommendation for the use of TM reagent.

TM reagent was added (200 ul) to 20 mg brain tissue in an Eppendorf vessel and homogenised at room temperature. Twenty microliters of 1-bromo-3-chloropropane were added and the Eppendorf was vortexed. After 10 min at room temperature it was centrifuged at 12,000×g for 15 minutes at 4° C. Then the colorless upper aqueous phase was removed to a fresh tube. 100 microliters of 2-propanol was added. Samples were centrifuged again 12,000×g for 10 minutes at 4° C. The supernatant was removed and the RNA pellet was washed with 200 ul cold 75% ethanol. The sample was re-centrifuged at 8000×g for 5 minutes at 4° C. The RNA pellet was dried for 5 minutes by air-drying on ice and 50 ul DEPC water were added to the RNA pellet. RNA was stored at −80 deg C. Genomic DNA strands were then removed by Turbo DNA-free Kit according to manufacturer's recommendation. RNA level was read by Nanodrop® reader. cDNA was prepared according to the manufacturer recommendation using High Capacity cDNA Reverse Transcription Kit (Applied Biosystems).

RT-PCR Reaction

RT-PCR reaction mix was prepared with Power SYBR® Green PCR Master Mix (Applied Biosystems) according to manufacturer instructions. Mixes were read with Mx300P (Stratagen) and the results of Ct were analysed according to the delta-delta Ct method of analysis. The value delta Ct is the difference between the Ct value of gene of interest to the Ct value of GAPDH normalizing gene. p-value <0.05 considered a significant alteration in mRNA expression.

TABLE 2

| Gene | Forward/ | Sequences |
|---|---|---|
| name | reverse | (5′-3′) |
| GAPDH | forward | AACTTTGGCATTGTGGAAGG |
| | reverse | ACACATTGGGGGTAGGAACA |
| MGL | forward | CAGAGAGGCCAACCTACTTTTC |
| | reverse | ATGCGCCCCAAGGTCATATTT |
| FAAH | forward | GGAAGTGAACAAAGGGACCA |
| | reverse | TCCCTGCAGCTTCAGTACCT |
| ABHD6 | forward | CCTTGATCCCATCCACCCCGGA |
| | reverse | CCCGGACACATCAAGCACCTGG |
| TNFα | forward | CAGGCGGTGCCTATGTCTC |
| | reverse | CGATCACCCCGAAGTTCAGTAG |
| IL-6 | forward | TAGTCCTTCCTACCCCAATTTCC |
| | reverse | TTGGTCCTTAGCCACTCCTTC |
| CB1 | forward | TCTTAGACGGCCTTGCAGAT |
| | reverse | AGGGACTACCCCTGAAGGAA |
| CB2 | forward | GAAACAGCCCGAGTCAGAAG |
| | reverse | GAGCCTGCCATTCTTACAGG |
| NAPE-PLD | forward | GCGCCAAGCTATCAGTATCC |
| | reverse | TCAGCCATCTGAGCACATTC |
| GAD67 | forward | CTCAGGCTGTATGTCAGATGTTC |
| | reverse | AAGCGAGTCACAGAGATTGGTC |

TABLE 3

| Protein | Gene |
|---|---|
| GAD67 | GAD1 |
| IL-6 | IL6 (human), I16 (rodents) |
| TNFalpha | TNF, Tnf |
| CB1 receptor | CNR1, Cnr1 |
| CB2 receptor | CNR2, Cnr2 |
| GPR55 | GPR55, Gpr55 |
| FAAH | FAAH, Faah |
| MGL | MAGLL, Magll |
| ABHD6 | ABHD6, Abhd6 |
| ABHD 12 | ABHD12, Abhd12 |
| ABHD4 | ABHD4, Abhd4 |
| DAGLalpha | DAGLA, Dagla |
| DAGLbeta | DAGLB, Daglb |
| NAPE-PLD | NAPEPLD, Napepld |
| GDE1 | GDE1, Gde1 |
| PLC | PLCB1, PLCB2, PLCB3, |
| | PLCD1, PLCE1, PLCG1, |
| | PLCG2, PLCZ1 |
| PLD | PLD3, PLD4 |
| 5-HT receptors | HTR, any HTR gene |

### Example 3

### Prophetic Example—Screening Subjects For Schizophrenia By DNA Microarray Analysis

Isolate DNA from primary cell culture from a patient or directly from a biological sample (biopsy) from a subject (epithelial cells, skin, saliva, lymphatic fluid, spleen, blood, urine, spleen, brain, spinal cord). Genotype humans using custom TaqMan single nucleotide polymorphism genotyping assay (Applied Biosystems) or custom genotyping arrays (e.g. MyGeneChip®, Affymetrix; Custom human array, Illumina). Include in the custom gene array probes for the genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR and combinations thereof. Perform assay according to manufacturer's instructions. Analyse results according to sex scored for each gene being compared. Anal Perform analysis in triplicate. Express results as mean fold change plus/minus standard error of mean. Compare results to healthy humans where population size is similar to the population size of schizophrenics with similar background (sex, age, ethnic origin etc.). Express results relative to housekeeping genes. The following conditions are required for a score to be significant: results should be within the detection limits of the gene array and/or p-value p<0.05 and/or average 1.5 fold-change.

### Example 4

### Prophetic Example—Diagnosing Humans For Schizophrenia, Diagnosing Humans for Schizophrenia by DNA Microarray Analysis

Perform diagnosis of an individual from DNA samples as above or from RNA samples. Isolate RNA from primary cell culture from a patient or directly from a biological sample from a patient (epithelial cells, skin, saliva, brain, spinal cord, lymphatic fluid, spleen, blood, urine). Take about 5 microgram total RNA to synthesize cDNA which is then used as a template to generate biotinylated cDNA. Genotype humans using custom TaqMan single nucleotide polymorphism genotyping assay (Applied Biosystems) or custom genotyping arrays (e.g. MyGeneChip®, Affymetrix; Custom human array, Illumina). Include in the custom gene array probes for the genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR and combination thereof. Perform assay according to manufacturer instructions. Results are analysed according to sex scored for each gene being compared. Analysis is performed in 2-3 repeats. Results are expressed as mean fold change plus/minus standard error of mean over a sample of 10 healthy humans and relative to housekeeping genes. The following conditions are required for a score to be significant: results are within the detection limits of the gene array and/or p-value p<0.05 and/or average 1.5-fold change.

### Example 5

### Prophetic Example—Diagnosing Humans for Schizophrenia by Real-Time PCR

Perform diagnosis of subject from RNA samples. Isolate the RNA from primary cell culture from a subject or directly from a biological sample from a subject (epithelial cells, skin, saliva, brain, spinal cord, lymphatic fluid, spleen, blood, urine). Extract RNA according to manufacturer's recommendation for the use of RNA extractions kits or TRI reagent. Add TRI reagent (200 ul) to 20 mg tissue and/or cells and homogenise at room temperature. Add 20 microliters of 1 bromo-3 chloropropane and vortex the Eppendorf. After 10 min at room temperature, centrifuge at 12,000×g for 15 minutes at 4° C. Then remove the colorless upper aqueous phase to a fresh tube. Add hundred microliter of 2-propanol. Centrifuge samples again 12,000×g for 10 minutes at 4° C. Remove the supernatant and wash RNA pellet with 200 ul cold 75% ethanol. Re-centrifuge the sample at 8000×g for 5 minutes at 4° C. Dry the RNA pellet for 5 minutes by air-drying on ice and add 50 ul DEPC water to the RNA pellet. Store RNA at −80 C. Remove genomic DNA strands by Turbo DNA-free Kit according to manufacturer recommendation. Read RNA level by Nanodrop® reader. Prepare cDNA according to the manufacturer recommendation using High Capacity cDNA Reverse Transcription Kit (Applied Biosystems). Prepare RT-PCR reaction mix with RT-PCR mix. Read with qPCR machine. Analyse results according to delta-delta Ct method.

### Example 6

### Prophetic Example—Personalised Medical Treatment—Screening Therapeutics for Individual Patient

Patients that have a mutation that disables the protein in one or more of the following genes encoding the proteins GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta , NAPE-PLD, GDE1, PLD, PLC, HTR and combination thereof cannot receive specific active agents. For example, do not treat with BCP a schizophrenic subject with a mutation in the CB1 receptor and/or CB2 receptor gene that result in absolute inactive CB1 receptor or CB2 receptor protein. Treat a schizophrenic patient with an active agent or a combination of active agents according to the results of a personalised gene sequencing (see below Example 7).

### Example 7

### Prophetic Example—Personalised Gene Sequencing

A genome sequencing is the operation in which the genome of an organism is completely or partially sequenced in laboratory to determine the specific DNA sequence of an organism's genome or specific proteins. This process reveals mutations including a single nucleotide polymorphism (SNPs). SNPs within the coding region can be of synonymous or nonsynonymous type. Nonsynonymous SNPs can change the amino acid sequence of protein whereas synonymous SNPs does not affect the protein sequence to the best of the current knowledge. SNPs which are not in protein-coding regions may still affect gene splicing, transcription factor binding, messenger RNA degradation, or the sequence of non-coding RNA. Gene expression affected by this type of SNP is referred to as an eSNP (expression SNP) and may be upstream or downstream from the gene.

Accordingly, determination of abnormal gene expression provides a signpost for therapeutic intervention and selected therapeutics can lead to inhibition of the expression of a specific gene or to enhancement of the expression of a specific gene.

For individual genome sequencing, isolate DNA or Circulating Cell-Free DNA (cfDNA) according to manufacturer instructions for example by Applied Biosystems and ThermoFisher Scientific. DNA and cfDNA are isolated from primary cell culture and/or from any biological sample of a human. DNA or cfDNA is isolated from a schizophrenic and/or PCP-addicted patient, for example but not limited to: epithelial cells, saliva, skin, brain, spinal cord, lymphatic fluid, spleen, blood, urine. The DNA sample is subjected to, for example, Next-Generation Sequencing technology and/or to Nanopore technology to obtain DNA sequence for the whole genome or DNA sequences at sites of interest for the genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR and combination thereof. Perform assay according to manufacturer's instructions. Analyse results for each gene and compare to the sequences of healthy humans. Results that underlie mutations or SNPs in the genome sequence of a patient are then diagnosed for effect of mutations and SNPs.

### Example 8

### Prophetic Example—Diagnosis of the Effect of Known Mutations

Diagnosis of the effect of known mutations can be extracted from current literature. Diagnosis of the effect of unknown mutations can be researched using cloning techniques where specific mutations are inserted into the DNA sequence of a desired protein. The DNA sequences are inserted into vectors and the vectors are expressed in cells for example but not limited to HEK293 and CHO cells. Alternatively, the DNA sequences are cloned into animals, e.g. to form a transgenic mice or a transgenic zebra fish, which expresses the specific mutations. DNA sequences of interest are for the genes encoding GAD67, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR and combination thereof. The activity of the mutant protein is then studied using routine assays such as pharmacological assays of binding and GTPγS binding, assays for testing the level of downstream effectors or downstream effects, genetic assays for the expression level of DNA, RNA of any kind. Accordingly, determination of abnormal activity of the mutant protein provides a signpost for therapeutic intervention and selected therapeutics to treat a specific schizophrenic or PCP-addicted patient.

### Example 9

### Prophetic Example—Personalised In Vitro/Cell Culture Response to Therapeutic Agents

In addition, or alternatively to cloning specific mutations, cells from a specific patient are isolated from, but not limited to, epithelial cells, saliva, skin, brain, spinal cord, lymphatic fluid, spleen, blood, urine and combination thereof, and grown in culture. The mutations in specific proteins are verified using DNA sequencing techniques as above for the genes encoding GAD67, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR combination thereof. Mutation-positive cells are grown and tested for the effect of the mutation according to known outputs. For example, a new mutation in the CB2 receptor gene can be investigated for the level of GTPγS activity using selective ligands such as HU-308 and JTE-907. Alternatively, the DNA sequences are cloned into animals, e.g. to form a transgenic mice or a transgenic zebra fish, which express the specific mutations. Accordingly, determination of abnormal activity of the mutant protein provides a signpost for therapeutic intervention and selected therapeutics to offer a specific schizophrenic, psychotic or PCP-addicted patient.

### Example 10

### Prophetic Example—Personalised Medical Treatment

Cells expressing a mutant DNA can be studied for their responses to selected therapeutics. Alternatively, the DNA sequences are cloned into animals, e.g. to form a transgenic mouse or a transgenic zebra fish, which express the specific mutations. Accordingly, determination of the response of cells expressing a mutant protein provides a signpost for therapeutic intervention and selected therapeutics to offer a specific schizophrenic, psychotic or PCP-addicted subject. Cells expressing mutations in the genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR and combination thereof. The effect of a selected therapeutics is studied according to technology. For example, the effect of BCP can be studied by GTPγS activity using selective ligands such as HU-308 and JTE-907. In another example, the effect of gene enhancers or antisense to the CB2 receptor gene (CNR2) is studied to alter the expression level of the CB2 receptor of a specific cell type.

### Example 11

### Prophetic Example—Genetic Tests of Cytochromes P450 (CYPSs) and their Function to Determine Best Combination of Therapeutic Agents

This step can be a complementary to the steps above for a successful use of the selected active agents. CYPs are the major enzymes involved in drug metabolism. Most active agents are degraded by CYPs, either directly or by facilitated excretion from the body. CYPS are responsible for about 75% of the total drug metabolism. Drugs may also increase or decrease the activity of CYPs. This can lead to treatment failure and to adverse drug reactions. For example, if one drug enhances the CYP-mediated metabolism of another drug, the second drug may not affect the patient. Another example is if one drug inhibits the CYP-mediated degradation of another drug, the second drug accumulates in the body and reaches toxic levels. Therefore, determining the genetic sequences of CYPS from DNA samples of a patient with schizophrenia or PCP-addicted is important for drug selection, establishing effective and successful treatment regime. Mutations in CYPs are determined by DNA sequencing and can be obtained while conducting the genomic sequencing or in addition to the selected genes encoding GAD67, IL-6, TNF-alpha, CB1 receptor, CB2 receptor, GPR55, FAAH enzyme, MGL enzyme, ABHD6, ABHD12, ABHD4, DAGL-alpha, DAGL-beta, NAPE-PLD, GDE1, PLC, PLD, HTR and combination thereof. In one example, a selected therapeutic is incubated with selected CYPs alone or in combination with other selected therapeutics according to the treatment regime for a specific patient. In another example, the mutant DNA sequence of CYP enzyme of a patient is expressed in cells and the selected therapeutics are incubated with the mutant CYPs to determine the activity of the mutant CYPs in the presence of the selected therapeutics. Therefore, determining the specific activity of a patient's CYPs in combination with selected therapeutics can prevent toxic and adverse reaction effects or reduced effect of the treatment regime due to enhancement of CYPs activity.

### Example 12

### Prophetic Example—Active Agent Treatment Selection

Mutations which are known to increase the expression level of any of the selected genes are then treated with antisense nucleic acids, ribozymes, small molecule promoter modulators, CB2 selective agonists, BCP, antibody, and combination thereof, to reduce the expression level of the selected genes.
a. Mutations which are known to reduce the expression level of any of the selected genes are then treated with gene enhancers, ribozymes, small molecule promoter modulators, CB2 selective agonists, BCP, HU-308, rosmarinic acid and combinations thereof.
b. Administrating BCP to alter expression of said gene; antibody, and combination thereof, to reduce the expression level of the selected genes.
c. Antisense oligonucleotides or nucleic acids
d. iRNAs (siRNA, miRNA, RITS)
e. Ribozymes
f. Gene enhancers DNA
g. Enhancer RNAs
h. Non-coding RNA transcripts
i. Small molecule promoter modulators.
j. CB2 selective agonists
k. BCP
l. HU-308
m. Rosmarinic acid
n. Antibody (whole antibody, humanized antibody, chimeric antibody, Fab fragment, Fab′ fragment, F(ab′)2 fragment, single chain Fv fragment and diabody)
o. Combination of a-n treatments

### REFERENCES

Atwood B K, Mackie K (2010). CB2: a cannabinoid receptor with an identity crisis. Br J Pharmacol 160: 467-479.
Coutts A A, Irving A J, Mackie K, Pertwee R G, Anavi-Goffer S (2002). Localisation of cannabinoid CB1 receptor immunoreactivity in the guinea pig and rat myenteric plexus. The Journal of Comparative Neurology 448: 410-422.
Hida H, Mouri A, Noda Y (2013). Behavioral phenotypes in schizophrenic animal models with multiple combinations of genetic and environmental factors. J Pharmacol Sci 121: 185-191.
Mouri A, Nagai T, Ibi D, Yamada K (2012). Animal models of schizophrenia for molecular and pharmacological intervention and potential candidate molecules. Neurobiol Dis.
Pertwee R G, Howlett A C, Abood M E, Alexander S P, Di Marzo V, Elphick M R, et al. (2010). International Union of Basic and Clinical Pharmacology. LXXIX. Cannabinoid receptors and their ligands: beyond CB(1) and CB(2). Pharmacol Rev 62: 588-631.
Ross C A, Margolis R L, Reading S A, Pletnikov M, Coyle J T (2006). Neurobiology of schizophrenia. Neuron 52: 139-153.
Weiser M, Noy S (2005). Interpreting the association between cannabis use and increased risk for schizophrenia. Dialogues Clin Neurosci 7: 81-85.

1. A method for screening and treatment of a mental disease selected from schizophrenia, psychosis and PCP abuse or addiction in a subject or a subject population, the method comprising:
a. screening a subject or a subject population for genetic mental disease susceptibility;
b. diagnosing in the subject or subject population the mental disease genetic susceptibility and at least one gene causing the mental disease susceptibility in a subject sample;
c. determining in the subject or subject population whether at least one of the genes causing the mental disease susceptibility has a gene mutation;
d. selecting out of a group of candidate active agents at least one active agent exhibiting activity in altering the expression of the at least one gene causing the mental disease susceptibility; and
e. treating the subject having the mental disease susceptibility with a therapeutically effective amount of the at least one active agent selected out of the group of candidate active agents or combinations thereof,
wherein treatment with a therapeutically effective amount of at least one active agent is gender-specific based on lateralization findings, and
wherein a subject having a gene mutation causing protein total inactivation in at least one of the genes leading to the mental disease susceptibility is not treated with the selected active agent.

2. The method of claim 1, wherein screening the subject or subject population for genetic mental disease susceptibility comprises determining in the subject sample the magnitude of expression of at least one gene causing the mental disease susceptibility in the subject or subject population, and comparing the magnitude of expression to a baseline magnitude of expression of the at least one gene, wherein departure from baseline magnitude of expression of at least one gene indicates the presence of the mental disease selected from schizophrenia, psychosis or PCP abuse or addiction.

3. The method of claim 1, wherein the at least one gene causing the mental disease susceptibility is selected from the group consisting of genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combinations thereof.

4. The method of claim 1, wherein the subject sample is harvested from a body fluid selected from cerebrospinal fluid (CSF), blood, saliva, lymphatic fluid, urine or feces, or from a body organ selected from epithelial cells, spleen, skin, hair or from a specific left or right side of the brain, prefrontal cortex, brain stem, hippocampus and/or spinal cord of a human or a nonhuman subject.

5. The method of claim 1, wherein diagnosing the mental disease susceptibility and the at least one gene causing the genetic susceptibility comprises screening, quantifying, visualizing, measuring the expression level and detecting departures from baseline of at least one gene selected from genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combinations thereof, using gene sequencing, PCR, RT-PCR, imaging systems, kits, arrays targeting DNA, RNA, protein in a whole body, a cell or a tissue sample harvested from a human or a nonhuman subject.

6. The method of claim 1, wherein the step of determining in the subject or subject population presence of the gene mutation leading to the mental disease susceptibility, comprises comparing in the subject sample the magnitude of expression of at least one gene causing the mental disease susceptibility and comparing the magnitude of expression to a baseline magnitude of expression of the gene, wherein altered gene expression indicates the presence of the mental disease, selected from schizophrenia, psychosis or PCP abuse or addiction.

7. The method of claim 1, wherein in the step of selecting out of the group of candidate active agents at least one active agent exhibiting activity in altering the magnitude of expression of the at least one gene causing the mental disease susceptibility, the at least one gene is selected from the group consisting of genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combinations thereof, wherein the selected active agent improves one or more symptoms of the mental disease selected from schizophrenia, psychosis or PCP abuse or addiction in a human or a nonhuman subject.

8. The method of claim 1, wherein the step of treating the mental disease in the subject or subject population comprises administering to a subject in need thereof the therapeutically effective amount of at least one selected active agent or combinations thereof, wherein the group of candidate active agents consists of a gene inhibitor selected from an antisense oligonucleotide, a nucleic acid molecule, an interfering RNAs (RNAi) selected from a small interfering RNA (siRNA), a micro interfering RNA (miRNA), an RNA-induced transcriptional silencing (RITS), a ribozyme and combinations thereof, a gene enhancer selected from a short DNA enhancer, an eRNA enhancer molecule and combinations thereof, a gene modulator selected from a non-coding RNA transcripts, a small molecule promoter modulators, a CB2 selective agonist selected from BCP and HU-308, a FAAH enhancer, a MGL enhancer, rosmarinic acid and combinations thereof, an antibody selected from whole antibody, humanized antibody, chimeric antibody, Fab fragment, Fab′ fragment, F(ab′)2 fragment, single chain Fv fragment, diabody and combinations thereof.

9. The method of claim 8, wherein administration to the subject in need thereof the therapeutically effective amount of the at least one selected active agent alters the magnitude of expression of at least one gene selected from the group consisting of genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combinations thereof, wherein the active agent is selected from:
a. a gene expression lowering amount of an antisense oligonucleotide, a siRNA, a ribozyme, a nucleic acid molecule or combinations thereof;
b. a gene expression enhancing amount of a gene enhancer, a nucleic acid molecule or combinations thereof;
c. a gene expression altering amount of at least one RNAi molecule or combinations thereof;
d. a gene expression altering amount of at least one a gene enhancer molecule or combinations thereof;
e. a gene expression enhancing amount of at least one non-coding RNA transcript or combinations thereof;
f. a gene expression altering amount of at least one RNA-cleaving ribozyme RNA or combinations thereof;
g. a gene expression altering amount of at least one small molecule promoter modulator or combinations thereof;
h. a gene expression altering amount of at least one CB2 selective agonist or combinations thereof;
i. a gene expression altering amount of BCP;
j. a gene expression altering amount of HU-308;
k. a gene expression altering amount of ABHD6 enhancer;
l. a gene expression altering amount of MGL enhancer;
m. a gene expression altering amount of FAAH enhancer;
n. a gene expression altering amount of rosmarinic acid;
o. a gene expression altering amount of an antibody selected from the group consisting of whole antibody, humanized antibody, chimeric antibody, Fab fragment, Fab′ fragment, F(ab′)2 fragment, single chain Fv fragment, diabody and combinations thereof;
p. combinations of above a-o active agents.

10. The method of claim 9, wherein the antibody specifically binds to an epitope of IL-6, TNF-alpha, CB1 receptor, CB2 receptor, GPR55, a 5-HT receptor or combination thereof prior to the manufacture of a medicament for the treatment of a mental disease selected from schizophrenia, psychosis and PCP abuse or addiction.

11. The method of claim 9, comprising reducing/increasing/stabilizing the amount of at least one protein encoded by at least one of the genes selected from the group consisting of genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combinations thereof by administration of a therapeutically effective amount of antibody or functional antibody fragment.

12. (canceled)

13. (canceled)

14. (canceled)

15. A method of screening for a candidate active agent for the treatment of a mental disease selected from schizophrenia, psychosis and PCP abuse or addiction comprising:
a. operatively linking a reporter gene which expresses a detectable protein to a regulatory sequence for a gene selected from the group consisting of genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combinations thereof, to produce a reporter construct;
b. transfecting a cell with the reporter construct;
c. exposing the transfected cell to a candidate active agent; and
d. comparing the level of expression of the reporter gene after exposure to the candidate active agent to the level of expression before exposure to the candidate active agent, wherein an alteration in the level of expression after exposure is indicative of the candidate active agent being useful for the treatment of a mental disease.

16. A kit comprising a custom array selected from a gene array, a probe array, a protein array, an array comprising a therapeutic agent, a nucleic acid molecule which selectively hybridizes to a nucleic acid molecule, a cell or a kit component which expresses a patient's mutation, to at least one of the genes selected from genes encoding GAD67, IL-6, TNF-alpha, CB1 receptor, CB2 receptor, GPR55, FAAH, MGL, ABHD6, ABHD12, ABHD4, DAGL-alpha, DAGL-beta, NAPE-PLD, GDE1, PLC, PLD, 5-HT receptors and combination thereof, and instructions for use it in a combination with other genes, proteins or combination thereof.

17. The method of claim 7, comprising selecting a CB2 selective receptor active agent for the treatment of a mental disease selected from schizophrenia, psychosis and PCP abuse or addiction, wherein selection is done in a native or constructed cell or in a transgenic/knockout animal expressing at least one 5-HT receptor or at least one mutant 5-HT receptor and combination thereof, by comparing the cell or animal response before and after exposure to the candidate active agent, wherein an altered level of response is indicative of suitability for the treatment of schizophrenia, psychosis and PCP abuse or addiction.

18. The method of claim 7 comprising determining gene expression or functional activity of a Cytochrome P450 enzyme in a homogenate mix, a cell, a mutant cell, a tissue or an organ originating from a human, an animal or a transgenic, knockout or conditional animal, wherein an alteration in the magnitude of the gene expression of the Cytochrome P450 enzyme activity or its gene expression is indicative of the active agent suitability for the treatment of schizophrenia, psychosis and PCP abuse or addiction.

19. The method of claim 1, wherein the subject is human or non-human animal.

20. The method of claim 1, wherein the mental disease is schizophrenia or psychosis and wherein said schizophrenia or psychosis includes any symptom and its onset is at any age.
